(19) United States
(12) Patent Application Publication
(10) Pub. No.: US 2025/0114517 A1
(43) Pub. Date: Apr. 10, 2025

(54) SYSTEMS AND METHODS FOR MONITORING PHYSIOLOGICAL STATUS OF LIVING SUBJECT AND ADMINISTERING SUBSTANCES THEREFOR

(71) Applicants: NORTHWESTERN UNIVERSITY, Evanston, IL (US); WASHINGTON UNIVERSITY, St. Louis, MO (US)

(72) Inventors: John A. Rogers, Wilmette, IL (US); Abraham Vazquez-Guardado, Evanston, IL (US); Joanna L. Ciatti, Evanston, IL (US); Yamin Zhang, Evanston, IL (US); Jin Wang, Evanston, IL (US); Robert W. Gereau, St. Louis, MO (US)

(21) Appl. No.: 18/567,428

(22) PCT Filed: Jun. 10, 2022
(86) PCT No.: PCT/US2022/033093

§ 371 Date: Dec. 6, 2023

Related U.S. Application Data
(60) Provisional application No. 63/209,057, filed on Jun. 10, 2021.

(51) Int. Cl. A61M 5/142; A61M 5/14; A61M 5/158; A61M 5/172; A61M 5/20; A61M 5/31; A61M 39/22; A61M 39/24; G16H 15/00

(52) U.S. Cl. CPC A61M 5/142; A61M 5/1408; A61M 5/1409; A61M 5/158; A61M 5/1723; A61M 5/2046; A61M 5/31; A61M 39/221; A61M 39/227; A61M 39/24; G16H 15/00; A61M 2005/14204; A61M 2005/206; A61M 2005/2073; A61M 2005/3114; A61M 2039/248; A61M 2202/0007; A61M 2202/04; A61M 2205/0216; A61M 2205/0233; A61M 2205/0238; A61M 2205/025; A61M 2205/04; A61M 2205/18; A61M 2205/3303; A61M 2205/3306; A61M 2205/3334; A61M 2205/3379; A61M 2205/3592; A61M 2205/502; A61M 2205/8206; A61M 2205/8237; A61M 2230/005; A61M 2230/04; A61M 2230/08; A61M 2230/20; A61M 2230/42; A61M 2230/50; A61M 2230/63

(57) ABSTRACT

An implantable device for monitoring a physiological status and administering drugs therefor includes at least one drug reservoir for containing at least one drug solution; a delivering member coupled to the at least one drug reservoir for operably delivering the at least one drug solution from the at least one drug reservoir to the living subject; a sensor member for measuring physiological parameters of a living subject so as to monitor a physiological status of the living subject; a wireless communication system for wireless data transmission; a power management system for wireless power harvesting; and a controller coupled to the power management system, the wireless communication system, the sensor member and the delivering member for wireless data transmission and power harvesting; obtaining the physiological status of the living subject, and controlling operations of the delivering member based on the physiological status of the living subject.

### CROSS-REFERENCE TO RELATED PATENT APPLICATION

This application claims priority to and the benefit of U.S. Provisional Application No. 63/209,057, filed Jun. 10, 2021, which is incorporated herein in its entirety by reference.

### STATEMENT AS TO RIGHTS UNDER FEDERALLY-SPONSORED RESEARCH

This invention was made with government support under grant numbers DA050303 and EB021793 awarded by the National Institutes of Health. The government has certain rights in the invention.

### FIELD OF THE INVENTION

The present invention relates generally to healthcare, and more particularly to systems and methods for monitoring physiological status of a living subject and administering substances therefor.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Over two million Americans are currently living with Opioid Use Disorders (OUD). Patients with OUD are at high risk of accidental overdose, which can produce respiratory depression leading to severe injury or death. We are in the midst of a well-publicized epidemic of deaths resulting from opioid overdose, with over 42,000 opioid-related deaths in the US in 2016. Currently, the treatment of opioid overdose requires the administration of the opioid receptor antagonist naloxone. Overdose reversal by naloxone is highly effective; however, successful deployment to an acutely overdosing patient requires (1) the timely identification that the patient is experiencing an overdose leading to dangerously low oxygen supply, (2) the immediate availability of naloxone, and (3) the means and skill on the part of the first responder or other party to administer the life-saving dose of naloxone via subcutaneous, intravenous, intramuscular, or intranasal administration. Given that overdoses can happen when the victim is alone and this overdose renders the individual incapacitated, injury and death are common outcomes. The current epidemic of opioid-related deaths ravaging the nation demands innovative new approaches to prevent deaths resulting from accidental overdose. The development of a fail-safe device to provide a life-saving dose of naloxone without the need for intervention by another party could significantly reduce opioid overdose related-mortality.

Patients with a history of opioid use followed by a period of sobriety are at particularly high risk for overdose. Prolonged use of opioids leads to the development of tolerance and the escalation of opioid dose used. Tolerance can quickly fade during a period of abstinence, so if a patient relapses and takes the same dose used prior to the period of abstinence, the dose will potentially be high enough to precipitate an acute respiratory crisis, leading to injury or death. Patients who are completing voluntary or compelled inpatient treatment for OUD are at particularly high risk. For these patients, it was concluded that “the elevated risk of dying from overdose within the first 4 weeks of leaving medication free inpatient treatment is so dramatic that preventive measures should be taken”. Individuals engaged with the criminal justice system are also at very high risk. People completing a period of incarceration or detention will similarly experience an extended period of sobriety. They also are not receiving (presumably) any treatment for their opioid use disorder during this period of detention, and are less likely to have a strong support system in place. They are therefore at extremely high risk of overdose when they are released from detention. For these individuals, innovative new interventions offer the possibility to decrease mortality dramatically.

Therefore, a heretofore unaddressed need exists in the art to address the aforementioned deficiencies and inadequacies.

### SUMMARY OF THE INVENTION

One of the objectives of this invention is to develop a novel implantable medical device that could offer a fail-safe approach to prevent accidental overdose deaths in these and other high-risk patient populations. The implantable device integrates micro-scale, wireless oximeters for measuring tissue oxygen saturation (rStO₂), fully implantable wireless microfluidic delivery systems, and closed-loop control hardware and software that enables delivery of a corrective stimulus in response to detection of pathological changes in physiology. The implantable device is a fully implanted, closed-loop system that detects decreased tissue oxygenation (an indicator of overdose and the cause of overdose fatality) and immediately delivers a life-saving dose of naloxone during an overdose without the need for intervention by first responders, and places automated calls for emergency assistance.

In one aspect of the invention, the device implantable for monitoring a physiological status of a living subject and administering drugs therefor includes at least one drug reservoir for containing at least one drug solution; a delivering member coupled to the at least one drug reservoir for operably delivering the at least one drug solution from the at least one drug reservoir to the living subject; a sensor member for measuring physiological parameters of a living subject so as to monitor a physiological status of the living subject; a wireless communication system for wireless data transmission; a power management system for wireless power harvesting; and a controller coupled to the power management system, the wireless communication system, the sensor member, and the delivering member for wireless data transmission and power harvesting; obtaining the physiological status of the living subject, and controlling operations of the delivering member based on the physiological status of the living subject.

In one embodiment, the device operably receives configuration commands and operation commands, wherein the configuration commands instruct the controller to deploy configuration parameters to the sensor member, or timing configuration for the delivering member, and wherein the operation commands trigger activation/deactivation of the sensor member or the delivering member at a time.

In one embodiment, the configuration commands and the operation commands are initialized from an external device in two-way wireless communications with the controller.

In one embodiment, the device is configured to receive inputs from the external device on patient condition or properties of the surroundings or other information streams, and to send data intermittently or continuously to the external device.

In one embodiment, the at least one drug solution comprises naloxone, or other life-saving drugs.

In one embodiment, the at least one drug solution enclosed in the at least one drug reservoir is releasable through outlets on the device, or through peripheral tubing.

In one embodiment, the drug release outlets are fitted with adapters to allow for facile connection to tubing for drug delivery.

In one embodiment, the adapters comprise Luer lock fittings.

In one embodiment, the at least one drug reservoir is refillable.

In one embodiment, the at least one drug reservoir comprises two or more drug reservoirs.

In one embodiment, each of the two or more drug reservoirs contains a same or different drug.

In one embodiment, a dose and a rate of the drug delivery from the two or more drug reservoirs is individually or cooperatively controllable.

In one embodiment, the dose of the drug delivery from the two or more drug reservoirs is determined based on the physiological status, weight, BMI, body temperature, and/or sex of the living subject.

In one embodiment, each of the two or more drug reservoirs has capacity to enclose about 0.1-3 mL of a drug.

In one embodiment, the delivering member comprises a wireless microfluidic delivery system.

In one embodiment, the delivering member is powered/operated by water electrolysis.

In one embodiment, the delivering member comprises a flexible membrane attached to the at least one drug reservoir; at least one electrolyte reservoir containing an aqueous electrolyte coupled to the flexible membrane; and at least one electrode coupled to the at least one electrolyte reservoir for producing the electrolysis of the aqueous electrolyte therein. The delivering member is configured such that during the water electrolysis, gas is formed in the at least one electrolyte reservoir, thereby increasing pressure of the at least one electrolyte reservoir, and as the pressure increases, it deforms the flexible membrane pushing the drug solution out of the at least one drug reservoir and into the surrounding tissue of the living subject.

In one embodiment, the electrolyte solution is an aqueous solution of alkali or alkaline earth metal hydroxide (i.e., KOH, NaOH).

In one embodiment, the electrolyte solution is an aqueous solution of alkali metal chloride salt (i.e., KCl, NaCl).

In one embodiment, the at least one electrode comprises copper interdigitated electrodes coated with a bilayer of nickel/gold or other metals such as platinum or nickel.

In one embodiment, the flexible membrane is formed of a flexible material.

In one embodiment, the flexible membrane can be formed by a single or multiple layers.

In one embodiment, the flexible material comprises polystyrene-b-polyisoprene-b-polystyrene (SIS), or similar block copolymers comprised of hard and soft blocks.

In one embodiment, the flexible membrane is a single-layered membrane, or a multilayered membrane.

In one embodiment, the flexible membrane is a smart membrane equipped with a sensing mechanism that enables monitoring of drug delivery rates.

In one embodiment, said monitoring of drug delivery rates is via strain sensing/deformation.

In one embodiment, the at least one electrolyte reservoir comprises an effervescent reservoir coupled to the flexible membrane; and at least one electrolyte chamber containing the aqueous electrolyte, wherein the at least one electrolyte chamber has a gate and is coupled to the effervescent reservoir, wherein the gate is configured to operably open or close the at least one electrolyte chamber such that when the gate is opened, a flow of the aqueous electrolyte into the effervescent reservoir is allowed, and when the gate is closed, the flow of the aqueous electrolyte into the effervescent reservoir is not allowed.

In one embodiment, the flexible membrane is attached to the effervescent reservoir.

In one embodiment, the gate is powered/operated by the electrolysis.

In one embodiment, the at least one electrolyte chamber is filled with citric acid.

In one embodiment, gas is formed in the at least one electrolyte chamber during the electrolysis, thereby increasing the pressure of the at least one electrolyte chamber, and as the pressure increases, it drives the gate to open and pushes the citric acid solution out of the at least one electrolyte chamber and into the effervescent reservoir.

In one embodiment, the effervescent reservoir is filled with sodium bicarbonate (NaHCO₃).

In one embodiment, the at least one drug reservoir comprises a low-friction hollow piston filled with the at least one drug solution.

In one embodiment, the delivering member further comprises a flexible film and a hollow needle attached to the hollow piston.

In one embodiment, the at least one drug solution is releasable through the hollow needle on the device.

In one embodiment, the citric acid reacts with sodium bicarbonate (NaHCO₃) once the gate opens and gas is generated in the effervescent reservoir, thereby increasing the pressure of the effervescent reservoir, and as the pressure increases, it pushes the flexible membrane and the low-friction hollow piston to a block position, while at the same time the fibrous capsule is punched through by the hollow needle, and the drug solution is pushed out from the low-friction piston into the surrounding tissue of the living subject.

In one embodiment, the device further comprises a cartridge module for deployment of the needle in the device.

In one embodiment, the cartridge module is actuated linearly to pierce nearby tissue with the needle or uses a rotational actuation to operate a blade for the deployment of the needle in the device.

In one embodiment, the cartridge module is configured such that the drug delivery mechanism produces hydraulic pressure behind a needle plunger which forces the needle through a protective septum and into the tissue, while simultaneously delivering the drug.

In one embodiment, the cartridge module is equipped with a permanent ring magnet and at least two solenoid coils, wherein a needle penetration force is generated by polarization of the at least two solenoid coils such that the at least two solenoid coils induce repulsive and attractive magnetic forces on the needle magnet, respectively.

In one embodiment, the needle is actuatable repeatedly for multiple piercing events or just once and retracted.

In one embodiment, the cartridge module includes one or more compressed springs for supplying penetration force upon triggering, wherein the one or more compressed springs are held in place with a triggerable stop pin, upon actuation, the one or more compressed springs spring release elastic energy to the needle and forces it into tissue.

In one embodiment, the cartridge module is a spring-loaded needle cartridge with an electromechanical triggering mechanism.

In one embodiment, the cartridge module is strategically located away from the body of the device in surrounding tissue to release drug remotely or integrated directly into the body of the device to allow for a more compact form factor, without requiring internal modification of the cartridge module.

In one embodiment, the drug delivery system comprises a plurality of microfluidic channel drug outlets in fluidic communications with the least one drug reservoir.

In one embodiment, the drug delivery system comprises one or more valves in fluidic communications with the least one drug reservoir for preventing leakage or accidental release of the drugs.

In one embodiment, the one or more valves are mechanical or passive valves, or pressure driven float/ball valves.

In one embodiment, the one or more valves comprise breakable seals, and/or elastic septum.

In one embodiment, the one or more valves are thermally or electrically activable.

In one embodiment, the drug delivery system comprises sheathed hollow needles to for piercing the fibrotic capsule during delivery to ensure fast dosage of rescue drug.

In one embodiment, the needles comprises microneedles or hypodermic.

In one embodiment, the drug delivery system comprises one or more sensors to monitor the fill level of drug and electrolyte reservoirs.

In one embodiment, the sensor member comprises at least one optical sensor.

In one embodiment, the sensor member comprises at least one photoplethysmography (PPG) sensor.

In one embodiment, the sensor member comprises a wireless oximeter for measuring regional tissue oxygen saturation (rStO2).

In one embodiment, the oximeter is assembled in the device, or is adapted as a peripheral probe.

In one embodiment, the sensor member further comprises one or more accelerometers for motion measurements, and/or one or more temperature sensors for temperature measurements, and/or ECG electrodes for electrocardiogram measurements.

In one embodiment, the sensor member is configured for multimodal sensing of parameters including SpO₂/StO₂, along with combinations of heart rate, heart rate variability, cardiac sounds, ECG, EMG, activity, body orientation, temperature, blood flow, blood pressure, respiratory rate, respiratory rate variability, respiratory effort/depth, blood chemistry, and subsets of the parameters.

In one embodiment, the sensor member comprises at least two temperature sensors separated by a distance to monitoring local thermal gradients for preventing tissue damage during battery charging.

In one embodiment, the device is configured to have separate and wirelessly connected components including an implant strategically located to measure SpO₂/StO₂ at an optimal body location, and a drug delivery device for drug delivery located at some other location optimized for that purpose.

In one embodiment, the drug delivery device is configured to deliver one or more drugs at one or more locations simultaneously or sequentially.

In one embodiment, the delivered amount of each of the one or more drugs at a respective one of the one or more locations is determined based on the detected physiological status of the living subject.

In one embodiment, the components are connected through physical means including wires, tubing, or mechanical structures.

In one embodiment, the components further comprises a battery that may be located separately from the other components of the device.

In one embodiment, the drug delivery device comprises a booster including integrating supercapacitors or other means to increase the peak power delivery capabilities for accelerating the rates of drug delivery.

In one embodiment, the drug delivery device comprises self-powered pumping mechanisms, wherein the system operably triggers the release of chemical energy through an exothermic chemical reaction, thereby ensuring proper, fast operation of the system even with a depleted battery.

In one embodiment, the wireless communication system comprises a near field communication (NFC) chip.

In one embodiment, the controller is operably in communications with the NFC chip and the sensor member via I2C communication protocol.

In one embodiment, power for the device is wireless transferred from an external radiofrequency (RF) power source and locally harvested on the devices.

In one embodiment, the power is locally harvested on the devices using a full-wave voltage rectifier, voltage regulator and a supercapacitor bank.

In one embodiment, the device is battery-free.

In one embodiment, the controller is configured to initialize measurement of the sensor member, collect the measured data therefrom, and then store the collected data in the NFC chip.

In one embodiment, an NFC reader connected to an external device and in communication with the NFC chip is adapted to provide RF power and communication to power and gain access the device, so as to deliver commands or extract the stored data from the NFC chip.

In one embodiment, a customized application with a graphic user interface (GUI) in the external device is adapted to control the flow of communication with the device via its configuration and operation commands.

In one embodiment, the NFC reader is further adapted to record the sensor member data and perform the data analytics and the closed loop logic of operation.

In one embodiment, the wireless communication system comprises a Bluetooth Low Energy (BLE) module.

In one embodiment, the power management system comprises a power module for providing power to the device.

In one embodiment, the power module comprises a battery and a battery charging module.

In one embodiment, the battery is a rechargeable battery.

In one embodiment, the battery charging module comprises a transdermal NFC wireless battery charging module.

In one embodiment, the battery charging module comprises a smart, intermittent charging algorithm to optimize battery charging while minimizing thermal dissipation at the electronics/tissue interface.

In one embodiment, the power management system is configured such that energy harvesting is from natural body motions, from thermal gradients, from biofuel cells, and the likes.

In one embodiment, the wireless communication system comprises both NFC and BLE communication protocols, optimized for low power operation.

In one embodiment, the controller is configured to control the Bluetooth communication with an external device; control the operation of the sensor member via I2C communication protocol; control the activation of each of at least one drug delivery pumps; and perform a hybrid power-up mechanism using a combination of firmware, software and passive NFC wireless power transfer, which allows the device to start up even when the battery has drained completely.

In one embodiment, a customized application with a graphic user interface (GUI) on the external device, such as a computer, smartphone or tablet, is adapted to establish and maintain BLE connection with the implanted device, control the flow of communication, perform data processing and closed loop logic of operation, log events, and trigger an emergency notice.

In one embodiment, the wireless communication system comprises a cellular or Wi-Fi link for emergency calls or other purposes.

This application contains two modes of operations accessible during device implantation at the clinic, and during device operation with the end user.

In one embodiment, the device has a size less than about 3.5 cm (length)×5 cm (width)×2 cm (height).

In one embodiment, the device further comprises an encapsulation layer that conformally coats entire surrounding of the device.

In one embodiment, the encapsulation is coated to prevent the formation of a fibrotic capsule by minimizing the foreign body response.

In one embodiment, the device is biocompatible.

In one embodiment, the device is formed to have smooth and rounded finishing.

In one embodiment, the device further comprises flap appendices that are used to secure the device with surgical sutures once implanted.

In one embodiment, the flap appendices are reinforced to prevent tear.

In one embodiment, the physiological parameters comprises at least one of a blood or tissue oxygenation, a heart rate, a respiratory rate, a temperature, an ECG, and a blood pressure.

In another aspect, the invention relates to a method for monitoring a physiological status of a living subject and administering drugs therefor. In one embodiment, the method includes continuously measuring physiological data of a living subject; processing the physiological data to extract physiological parameters including a heart rate and/or a tissue oxygenation; determining whether the tissue oxygenation monotonically drops over a period of time based on the physiological parameters; and in the event of an overdose when the tissue oxygenation is lower than a baseline level over the period of time, administering a dose of a drug to the living subject.

In one embodiment, each of the measuring step and the administering step is performed by a device implanted in the living subject, and wherein each of the processing step and the determining step is performed by an external device that is in two-way wireless communication with the device.

In one embodiment, the method further comprises, prior to the processing step, transmitting, by the device, the physiological data to the external device.

In one embodiment, the method further comprises receiving inputs from the external device on patient condition or properties of the surroundings or other information streams; and sending the measured physiological data intermittently or continuously to the external device.

In one embodiment, the method further comprises generating a report of events, by the external device, that include time of event; doses self-administered by the device; the levels of oxygenation recorded before, during, and after the overdosing event; and/or geolocation data to be used as a localization resource.

In one embodiment, the method further comprises triggering an emergency notice with geolocation data to first responders in the event of the overdose.

In one embodiment, the method further comprises providing power to the device via a battery.

In one embodiment, the method further comprises wirelessly charging the battery.

In one embodiment, the method further comprises monitoring and controlling safe charging of the battery based on thermal thresholding detected from temperature sensors.

In one embodiment, the method further comprises optimizing operations of the device to prolong battery lifetime and capacity.

In one embodiment, the method further comprises providing power to the device via an NFC chip for power harvesting wirelessly.

In one embodiment, the dose of the at least one drug is determined based on the physiological status, weight, BMI, body temperature, and/or sex of the living subject.

In yet another aspect, the invention relates to a method for operating continuously and autonomously using hardware/firmware embedded in the implantable device comprising: recording data into memory; making decisions on drug release; generating a report of events; and triggering information transfer and/or emergency calls.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows schematically an implantable device with an external link according to one embodiment of the invention. Panel A: Implantable device: fluidic pumps for drug delivery, oximeter, NFC communication. Panel B: External link: data processing and event logging, data display, feedback commands to device.

FIG. 2 shows schematically a closed-loop logic block diagram of an implantable device according to one embodiment of the invention. Panel A: the implantable device. Panel B: external data processing. Panel C: method for delivering an emergency call to a receiver (e.g., emergency responders).

FIG. 3 shows schematically device/software operation workflow for oximeter of an implantable device according to one embodiment of the invention. Battery-free operation requires power management schemes, hardware, and software synchronization to achieve continuous recording.

FIG. 4 shows data acquired from a human finger resting on a wireless oximeter according to one embodiment of the invention.

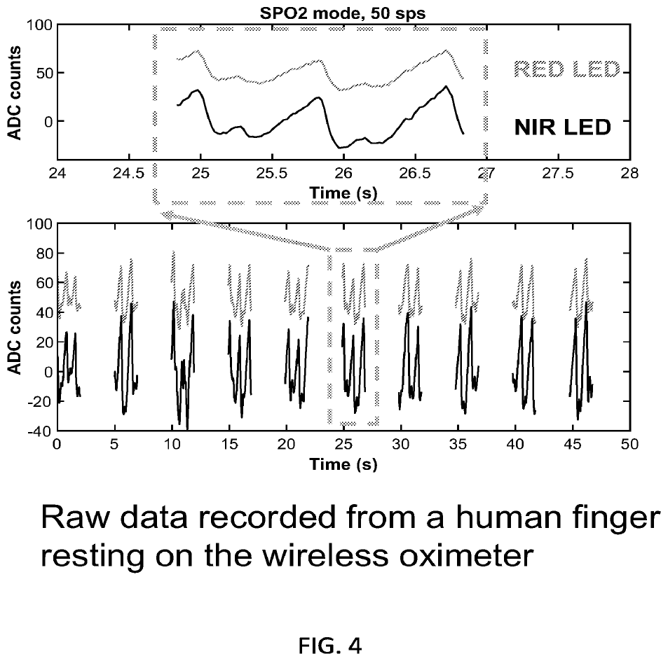

FIG. 5 shows a customized graphic user interface (GUI) for drug delivery and oximeter control according to one embodiment of the invention. Graphic user interface controls: configuration/operation of the oximeter, configuration/operation of the fluidic pumps for drug delivery, data analysis in real time, algorithm for closed-loop operation and access via Bluetooth to a peripheral phone for emergency call.

FIG. 6 shows schematically an implantable device according to one embodiment of the invention. Panel A: an exploded view of multiple components that construct the device. The multiple components include, but are not limited to, microfluidic channels, drug exits ports, two 150 μL drug reservoirs, a flexible membrane, two electrolyte reservoirs, electrodes and electronics PCB. Panel B: Photograph of the device.

FIG. 7 shows schematically life expectancy of electrodes of an implantable device according to one embodiment of the invention. Electrode degradation due to corrosion caused by the electrolyte (KOH 50 mM) at 75° C. for 5 weeks is negligible. This gives a projected electrode lifetime of at least 6 months. Panel A: Photograph of pristine electrodes. Panel B: Activation current measured over 5 weeks. Panel C: Photograph of electrodes after 5 week incubation in 50 mM KOH at 75° C.

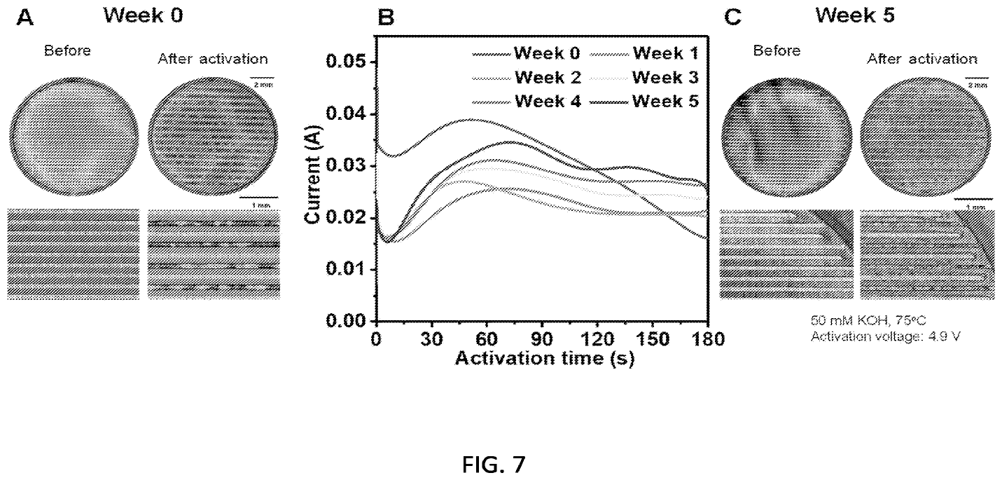

FIG. 8A shows schematically the mechanism for drug release via water electrolysis according to one embodiment of the invention.

FIG. 8B shows schematically structure of interdigitated electrodes according to one embodiment of the invention.

FIG. 9 shows respiratory depression in vivo captured by the optical oximeter of an implantable device according to one embodiment of the invention. Panel A: Raw data recorded by the sensor during respiratory depression. Panel B: Comparison between the implanted device and a commercial oximeter. Panel C: Optical absorption of Hemoglobin at two states; oxygenated hemoglobin (HbO₂) and deoxygenated hemoglobin (Hb).

FIG. 10 shows closed-loop control of respiratory depression using an implantable device according to one embodiment of the invention. Panel A: Wireless oximeter detects the drop of oxygenation due to fentanyl injection, and then triggers the pump and emergency call. Panel B: Photograph of a rat with a subdermal device. Panel C: Tissue oxygenation recorded by the device showing the event at which it triggers the release of naloxone and the emergency call. Panel D: Commercial oximeter confirms the dynamics of the respiratory depression.

FIG. 11 shows oxygenation traces demonstrating respiratory depression and recovery acquired by an implantable device according to one embodiment of the invention. Panel A: Graph comparing the SpO₂ recovery dynamics in-vivo after Fentanyl injection in the following cases: 1) Without naloxone injection, 2) With manual subcutaneous naloxone injection, and 3) With automated naloxone delivery by the implantable device. Panel B: Graph shows the elapsed time for the animal model to recover 90% SpO₂. Closed loop administration of Naloxone produces comparative recovery as subcutaneous manual injection of naloxone.

FIG. 12 shows schematically an implantable device according to one embodiment of the invention. The device contains two 1 mL drug reservoirs powered by electrolytic pumps, a rechargeable battery, and a BLE oximeter for communication and control. Panel A: an exploded view of the device. Panels B-C: Photographs of one prototyped device.

FIG. 13 shows a BLE implantable oximeter and acquired PPG signals according to one embodiment of the invention. Panel A: BLE implantable oximeter: 50 samples per second oximeter, 16 bit resolution, continuous oximeter data transmission for at least 20 hrs, and battery wireless charging module. Panel B: Photograph of a human finger on the wireless oximeter sensor.

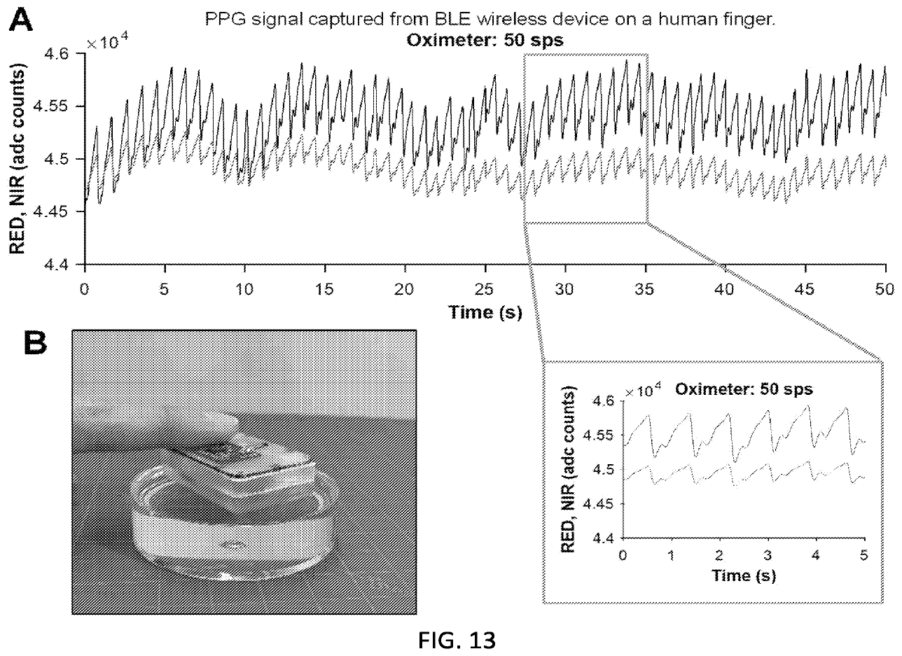

FIG. 14 shows an application for communication and control according to one embodiment of the invention. Screen shot of an iPad application that provides access to device configuration and control, Right panel show the raw data being recorded from the device in real time.

FIG. 15 shows schematically a closed-loop logic block diagram of an implantable device according to one embodiment of the invention. Panel A: Battery operation with wireless battery recharging module. Panel B: The implantable device contains a BLE ship, 1-3 mL of Naloxone solution, optical oximeter, wireless power harvesting, and battery and power management. Implementation of a dedicated power management scheme enables continuous operation on a single battery charge. Panel C: Implanted device establishes and maintain a BLE connection with smart phone or tablet that performs data analytics, and triggers drug release and emergency call. Panel D: Emergency call delivered from the smartphone or tablet to a receiver, e.g., first responders, via cellular network.

FIG. 16 shows schematically exploded view illustrations of designs for a miniaturized, implantable oximeter and drug delivery system according to one embodiment of the invention, for use implanted in the abdominal region of a rat. Panel A: Catheter-type oximetry sensing probe. Panel B: Microfluidic system for programmable pharmacology. Panel C: Placement of the integrated system.

FIG. 17 shows schematically the circuit diagram of the wireless control and power module according to one embodiment of the invention. Panel A: Operational block diagram of the overall system design. BLE, Bluetooth Low Energy. Panel B: Power management circuitry.

FIG. 18 shows schematically algorithm for robust, reliable measurement of rStO₂, according to one embodiment of the invention. Panel A: Flow chart of the architecture for evaluating a signal quality index (SQI) based on a neural network classification method. Panel B: Block diagram of processing method for determining rStO₂, indicated generally as SpO₂.

FIG. 19 shows schematically closed-loop operation according to one embodiment of the invention. Flow chart of the steps in the closed-loop operation for drug delivery, starting with collection of data from the μ-IPD (indicated here as PD) when oximetry level falls below a threshold three times in a row across a set time.

FIG. 20 shows an exemplary example utilizing the system of the invention for an animal model experiment. Panel A: Schematic representation of the behavioral methodology. Briefly, rats trained to self-administer sucrose will undergo surgery to implant the jugular catheter with or without implantation of and either the noninvasive pulse oximeter collar or our wireless closed-loop device. In some rats the noninvasive pulse oximeter collar will be used. A week after recovery animals will be able to self-administer i.v. fentanyl (2 g/kg/infusion) during a 2 hr session. Panel B: Picture of a freely moving rat self-administering fentanyl with non-invasive pulse oximeter collar. Panel C: Response curve across days for fentanyl (2 g/kg/infusion) i.v. self-administration in rats. Fixed Ratio (FR) numbers represent the number of active lever presses to obtain a single infusion. Animals maintain a steady fentanyl consumption across days despite increasing effort required to obtain the same dose. Panel D: Representative trace of SpO₂ obtained using non-invasive pulse oximeter collar. A significant decrease in SpO₂ is observed 10 min after an i.v. bolus injection of Fentanyl (20 g/kg) and reversed after i.v. administration of Naloxone (1 mg/kg).

FIG. 21 shows schematically an implantable device according to one embodiment of the invention. Panel A: an exploded view of multiple components that construct the device. The multiple components include, but are not limited to, a drug reservoir, a flexible membrane, an effervescent reservoir chamber filled containing NaHCO₃, a gate, an electrolyte chamber filled with citric acid, and electrodes. Panel B: an assembly of a gate and an electrolyte chamber of the device showing the gate in an open state.

FIG. 22 shows schematically an implantable device according to one embodiment of the invention. Panel A: an assembly of the device. Panel B: Photograph of the device.

FIG. 23 shows electrical current of the electrolysis at various applied voltages according to one embodiment of the invention.

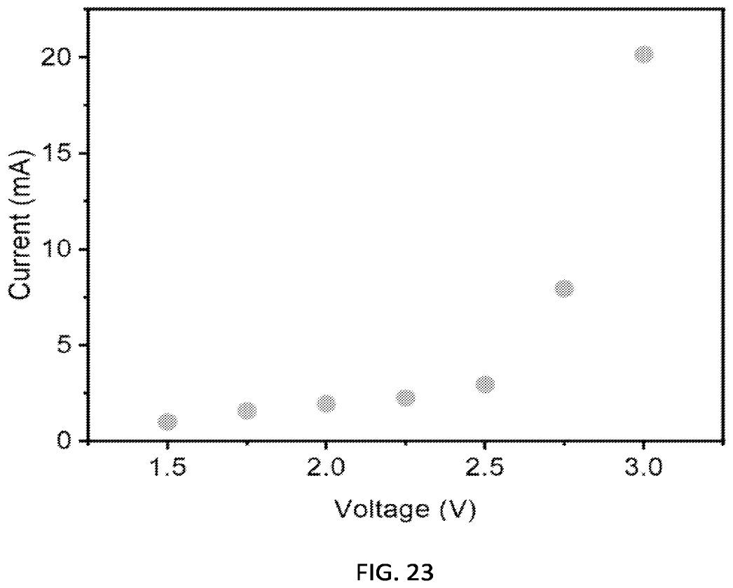

FIG. 24 shows a relationship of membrane expanding vs time according to one embodiment of the invention.

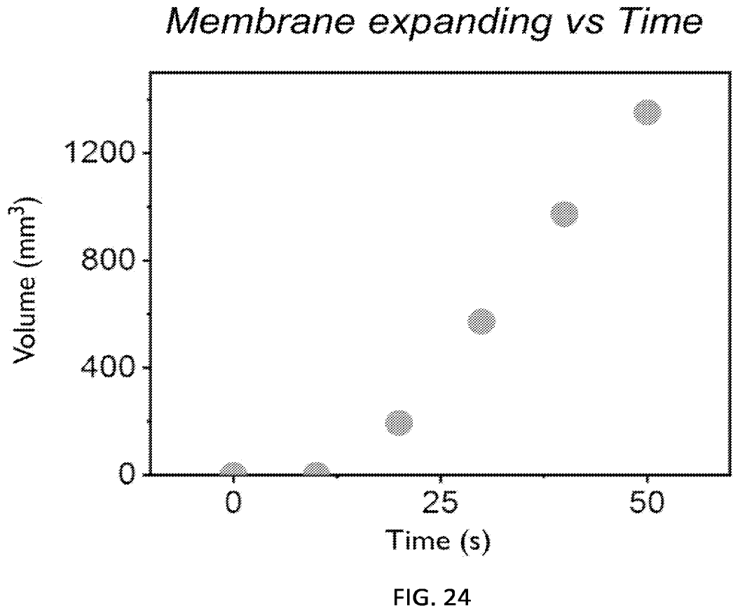

FIG. 25 shows time needed to release 0.5 ml drug according to one embodiment of the invention.

FIG. 26 shows experimental data for tissue oximetry during in-vivo calibration experiment with the implantable oximeter according to embodiments of the invention and comparison with standards. Panel A: Oximetry (StO₂) in porcine models during inspired oxygen modulation in-vivo calibration experiment using the implantable device equipped with an oximeter sensor and commercial tissue oximeter (StO₂—Cutaneous Tissue Ox) and standard blood gas (Blood gas). Panel B: Correlation curve showing good linearity of the implantable oximeter compared with blood gas (70% Venous:30% Arterial); Correlation coefficient=0.9 (R²), slope of linear regression=0.4. Panel C: Temporal spectrogram that shows the frequency component that corresponds to the heart rate (˜100 bpm) and respiration rate (˜20 Bpm) during the experiment. Panel D: Raw data extracted from the implantable oximeter that show the red and near-infrared (NIR) light channels recorded from the photodiode. The oscillations show the presence of respiration and cardiac pulsation.

FIG. 27 shows probe designs that contain an oximeter for tissue oximetry according to embodiments of the invention. Panels A-C: Planar probe design comprised of polyimide/copper/polyimide/copper/polyimide flexible interconnects (thicknesses 12.5/12/25/12/12.5 μm) that join the oximeter sensor with the device body. Panel D: Cylindrical probe containing the oximeter sensor interconnected with flexible multistranded copper wires to the body of the device.

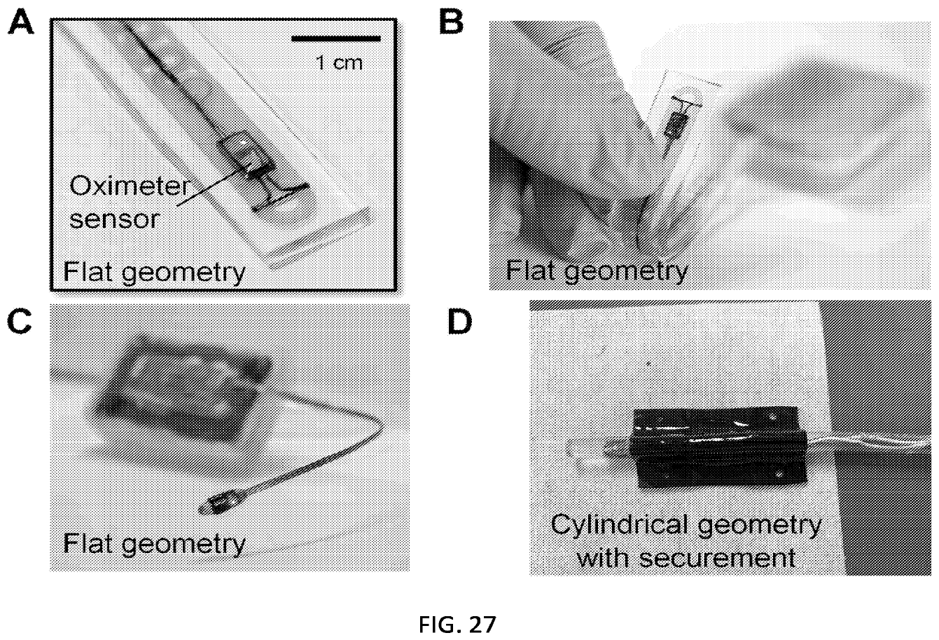

FIG. 28 shows schematically two actuation mechanisms to rupture the fibrous capsule tissue during chronic device implantation according to embodiments of the invention. Panel A: Spring-loaded needle linear actuation for piercing fibrous capsule and deliver drug in the surrounding tissue. Panel B: Spring-loaded blade configuration to slice fibrous tissue and allow drug absorption in the surrounding tissue.

FIG. 29 shows schematically a needle actuation mechanism powered by hydraulic pressure according to embodiments of the invention. Panel A: schematic showing the system in the resting state. Panel B: Schematic showing the system after actuation. Hydraulic pressure accumulates exerting pressure on the needle plunger.

FIG. 30 shows schematically a needle actuation mechanism powered by magnetic force according to embodiments of the invention. Panel A: The needle plunger is equipped with a permanent ring magnet, and the needle cartridge is equipped with two solenoids. Panel B: During forward activation, Coil 1 exerts a repulsive force to the magnet whilst Coil 2 exerts an attractive force. Panel C: During backward activation, Coil 2 exerts a repulsive force to the magnet whilst Coil 1 exerts an attractive force.

FIG. 31 show schematically a spring-loaded needle linear actuator triggerable with heat. Panel A: The system comprises a compression spring engaged and secured with a stop mechanism. Panel B: A heating device activates the thermal trigger releasing the mechanical force from the spring and launching the needle into tissue.

FIG. 32 show schematically a spring-loaded needle linear actuator triggerable with a combined magnetic and mechanical trigger according to embodiments of the invention. Panel A: The needle cartridge is equipped with a compressed spring and a stop pin secured with a safety spring that holds the pin in place. Panel B: Activating a solenoid generates a magnetic force on the permanent magnet, compressing the safety spring and releasing the needle.

FIG. 33 show schematically two embodiments of an implantable device equipped with a modular linear needle actuator cartridge according to the invention. Panel A: The needle actuator cartridge located separate from the body of the device at the tip of a drug catheter. Panel B: The needle actuator cartridge located within the body of the device.

FIG. 34 show schematically examples of the implantable device containing a set of anchoring points for securing the devices to tissue while implanted according to embodiments of the invention.

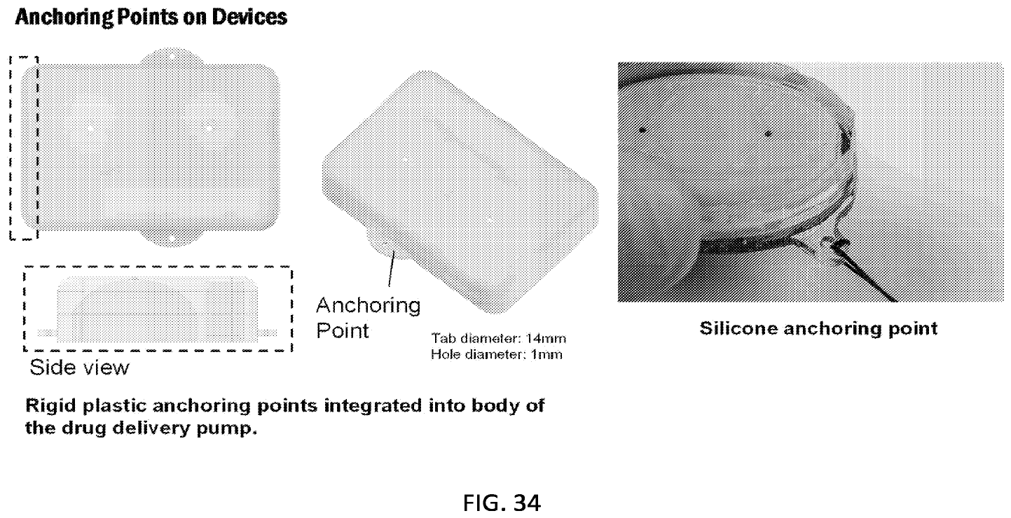

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in this disclosure, the term “living subject” refers to a living human or a living animal. Devices and methods of this invention are primarily designed for offering a fail-safe approach to prevent accidental drug overdose deaths in high-risk populations of patients (living human). For the purpose of illustration of the invention, the devices and methods are applied to monitor the physiological status of rats and administer drugs therefor based on the monitored physiological status, which is in no way intended to limit the invention, its application, or uses.

The terms “flexible” and “bendable” are used synonymously in the present description and refer to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. As used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and mesh geometries.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

One of the objectives of this invention is to provide a novel implantable device that could offer a fail-safe approach to prevent accidental drug overdose deaths in high-risk patient populations.

In certain aspects of the invention, the implantable device comprises an implantable, closed-loop system that senses the presence of dangerously low tissue oxygenation that results from a drug overdose (e.g., opioid overdose), and automatically administers a life-saving bolus injection of naloxone or other life-saving substances. The device utilizes various communication protocols to automatically alert family and first responders to the presence of an overdose event. The implantable device in certain embodiments integrates micro-scale, wireless oximeters for measuring tissue oxygen saturation (rStO₂), fully implantable wireless microfluidic delivery systems, and closed-loop control hardware and software that enables delivery of a corrective stimulus in response to detection of pathological changes in physiology. The implantable device is a fully-implanted, closed-loop system that detects decreased tissue oxygenation that is an indicator of overdose and the cause of overdose fatality and immediately delivers a life-saving dose of naloxone during an overdose without the need for intervention by first responders, and places automated calls for emergency assistance. The implantable device is biocompatible and formed to have smooth and rounded texturing finishing.

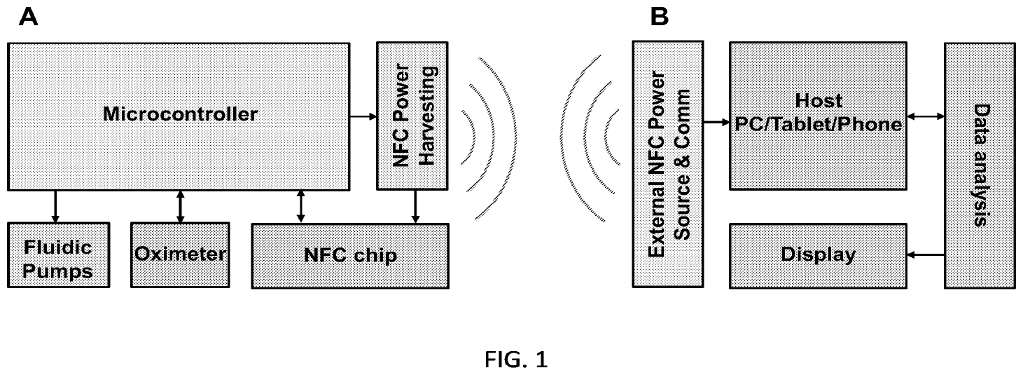

In some embodiments, as shown in FIGS. 1, 6, 12 and 15-16, the implantable device includes at least one drug reservoir for containing at least one drug solution; a delivering member coupled to the at least one drug reservoir for operably delivering the at least one drug solution from the at least one drug reservoir to the living subject; a sensor member for measuring physiological parameters of the living subject so as to monitor a physiological status of the living subject; a wireless communication system for wireless data transmission; a power management system for wireless power harvesting; and a controller coupled to the power management system, the wireless communication system, the sensor member, and the delivering member for wireless data transmission and power harvesting; obtaining the physiological status of the living subject, and controlling operations of the delivering member based on the physiological status of the living subject. The physiological parameters comprises at least one of a blood oxygenation, a heart rate, a respiratory rate, a temperature, and a blood pressure. In some embodiments, the at least one drug solution comprises naloxone, or other life-saving drugs.

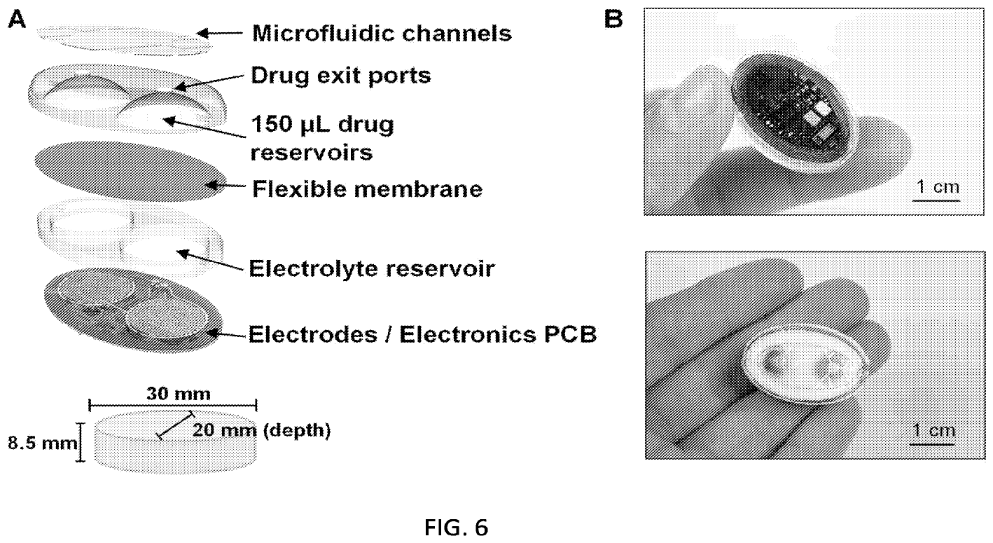

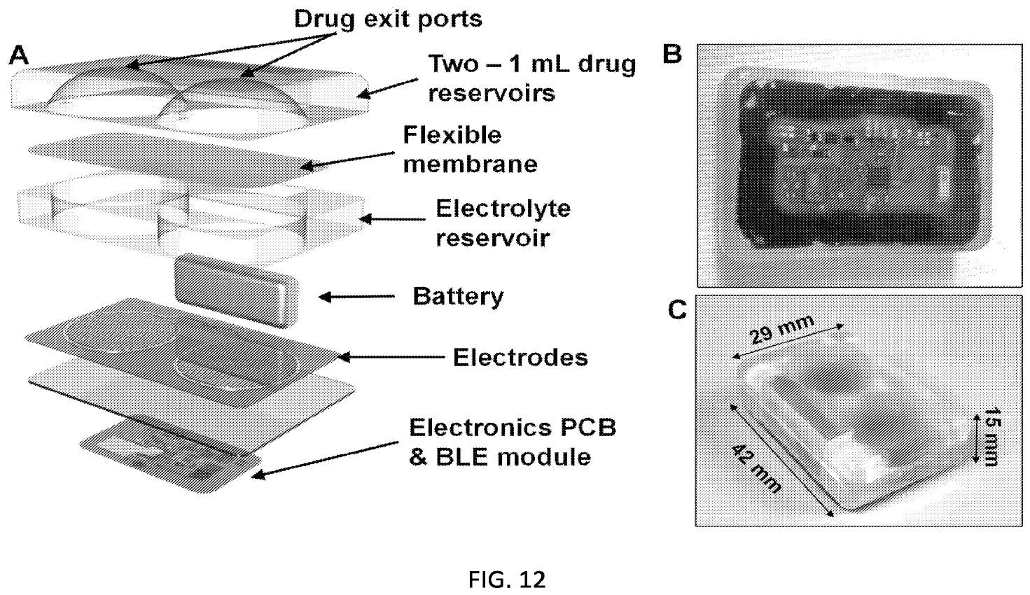

In some embodiments, the at least one drug reservoir comprises a single reservoir. In other embodiments, the at least one drug reservoir comprises two or more drug reservoirs, with each drug reservoir containing a same or different drug. In some embodiments, each drug reservoir has capacity to enclose about 0.1-3 mL of a drug. As shown in FIGS. 6 and 12, the device includes two drug reservoirs. Each reservoir has the capacity to enclose about 150 μL drug (FIG. 6), or about 1 mL drug (FIG. 12). In some embodiments, the at least one drug reservoir is refillable.

In some embodiments, the at least one drug solution enclosed in the at least one drug reservoir is releasable through outlets or exit ports on the device, or through peripheral tubing. In some embodiments, the drug release outlets are fitted with adapters to allow for facile connection to tubing for drug delivery. In some embodiments, the adapters comprise Luer lock fittings.

In some embodiments, a dose and a rate of the drug delivery from the two or more drug reservoirs is individually or cooperatively controllable. In some embodiments, the dose of the drug delivery from the two or more drug reservoirs is determined based on the physiological status, weight, BMI, body temperature, and/or sex of the living subject.

In some embodiments, the delivering member (drug delivery system), comprises a wireless microfluidic delivery system, such as fluidic micropumps.

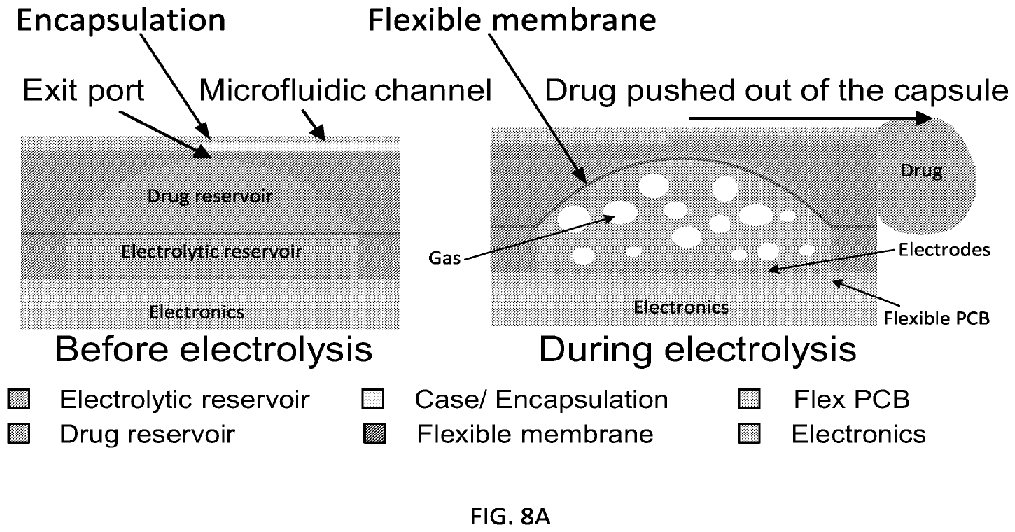

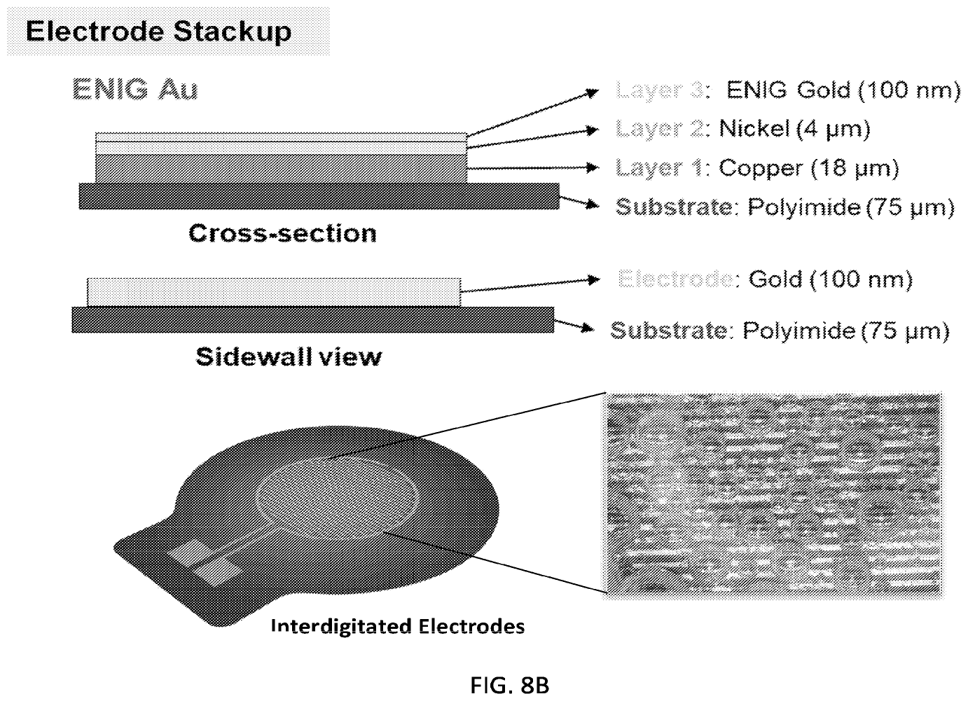

In some embodiments, the delivering member is powered/operated by gas-producing water electrolysis, which is a pure electrochemical process. For example, as shown in FIGS. 6, 8, 12 and 16, the delivering member comprises a flexible membrane attached to the at least one drug reservoir; at least one electrolyte reservoir containing an electrolyte coupled to the flexible membrane; and at least one electrode coupled to the at least one electrolyte reservoir for producing the electrolysis of the electrolyte therein. In addition, the delivering member may further include electronics formed on a printed circuit board (PCB) for activating/driving the at least one electrode for electrolysis (FIGS. 6, 8 and 12). The delivering member is configured such that during the electrolysis, gas is formed in the at least one electrolyte reservoir, thereby increasing pressure of the at least one electrolyte reservoir, and as the pressure increases, it deforms the flexible membrane pushing the drug solution out of the at least one drug reservoir and into the surrounding tissue of the living subject, as shown in FIG. 8.

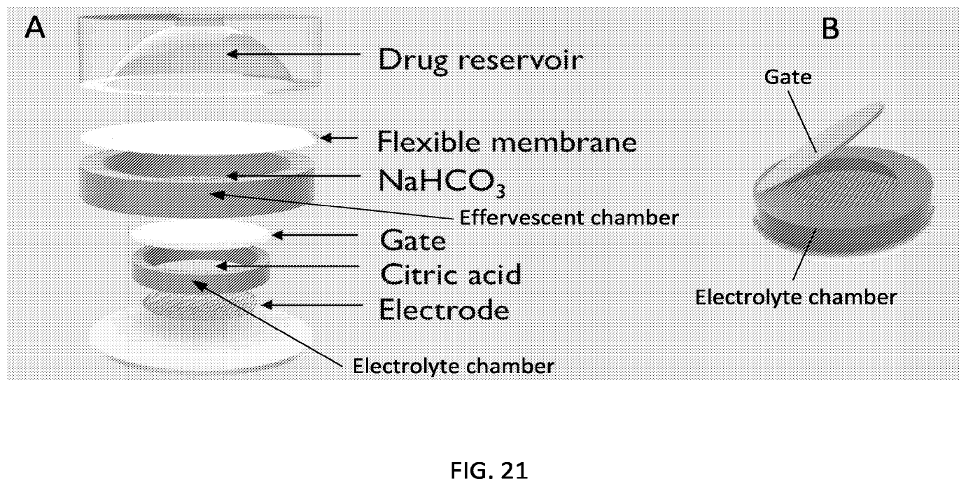

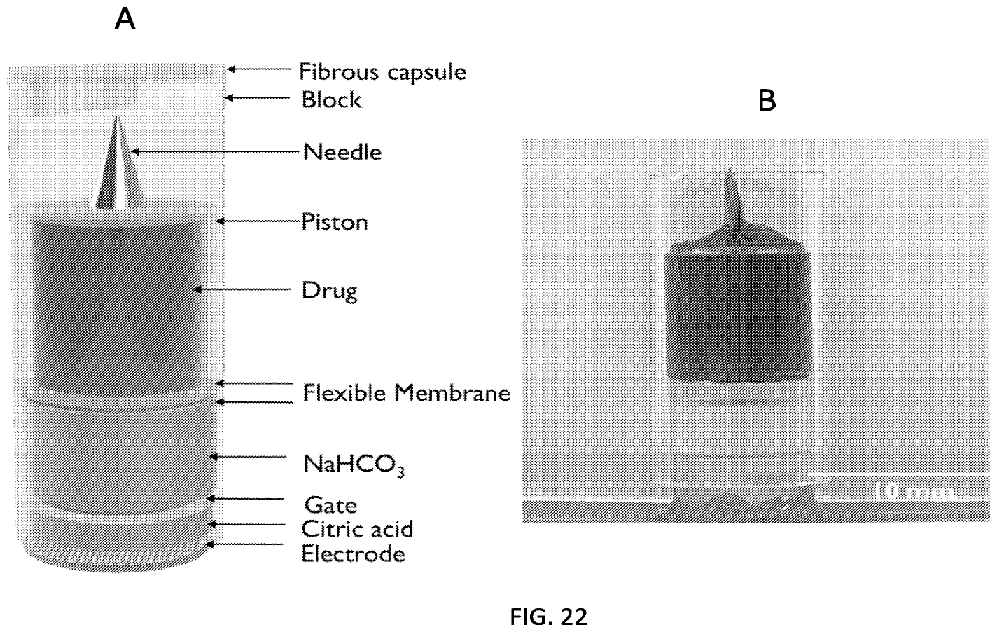

In some embodiments, the delivering member is powered/operated by both water electrolysis and chemical reaction, which are electrochemical and chemical processes. As shown in FIGS. 21-22, the electrolyte reservoir comprises an effervescent reservoir coupled to the flexible membrane; and at least one electrolyte chamber containing the aqueous electrolyte. In some embodiments, the flexible membrane is attached to the effervescent reservoir which in some embodiments is filled with sodium bicarbonate (NaHCO₃).

The at least one electrolyte chamber has a gate and is coupled to the effervescent reservoir. The gate is configured to operably open or close the at least one electrolyte chamber such that when the gate is opened, a flow of the aqueous electrolyte into the effervescent reservoir is allowed, and when the gate is closed, the flow of the aqueous electrolyte into the effervescent reservoir is not allowed. In some embodiments, the at least one electrolyte chamber is filled with citric acid. In some embodiments, the gate is powered/operated by the electrolysis.

In some embodiments, the at least one drug reservoir comprises a low-friction hollow piston filled with the at least one drug solution (FIG. 22). In some embodiments, the delivering member further comprises a flexible film and a hollow needle attached to the hollow piston. In some embodiments, the at least one drug solution is releasable through the hollow needle on the device. In some embodiments, as discussed in EXAMPLE 12, different actuated mechanisms can be utilized to deploy the needle to deliver the drug.

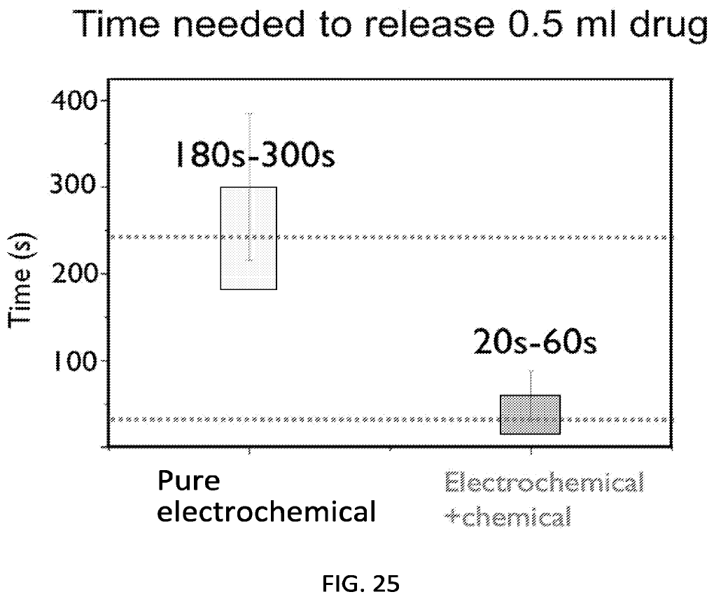

In operation, gas is formed in the at least one electrolyte chamber during the electrolysis, thereby increasing the pressure of the at least one electrolyte chamber, and as the pressure increases, it drives the gate to open and pushes the citric acid solution out of the at least one electrolyte chamber and into the effervescent reservoir, where the citric acid reacts with sodium bicarbonate (NaHCO₃) in the effervescent reservoir chamber. Such a chemical reaction can generate more gas in a very short time, thereby increasing the pressure of the effervescent reservoir in the effervescent reservoir chamber quickly. As the pressure increases, it pushes the flexible membrane and the low-friction hollow piston to a block position, while at the same time the fibrous capsule is punched through by the hollow needle, and the drug solution is pushed out from the low-friction piston into the surrounding tissue of the living subject. As shown in FIG. 25, the time needed to release about 0.5 ml drug is about 20-60 seconds for the device shown in FIGS. 21-22 operated by both the electrochemical electrolysis and chemical reaction, while the time needed to release about 0.5 ml drug is about 180-300 seconds for the device shown in FIGS. 6 and 12 operated by the electrochemical electrolysis.

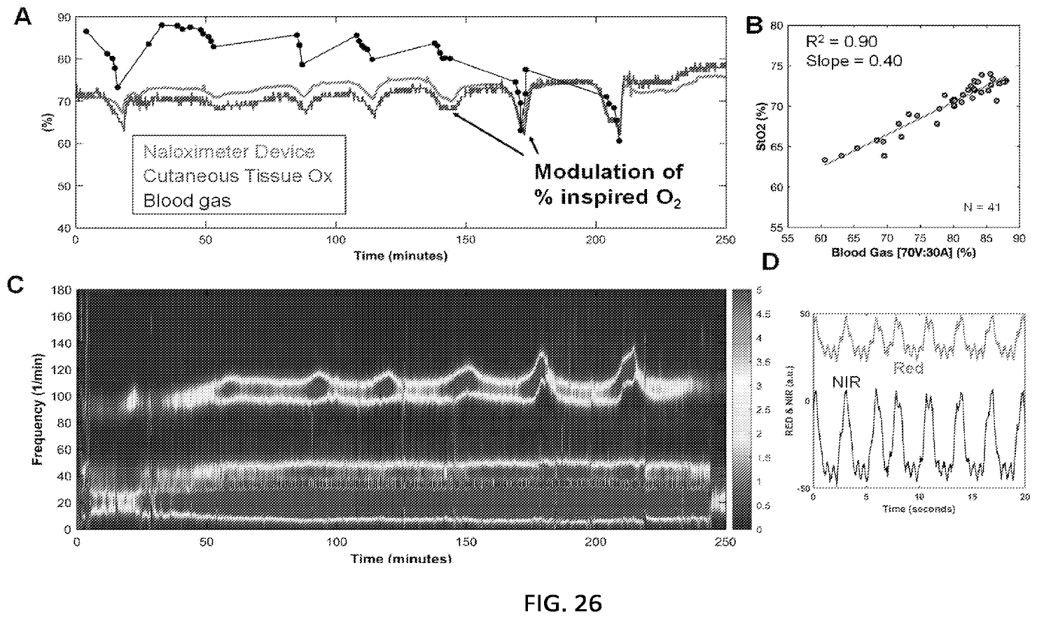

FIG. 26 shows experimental data for tissue oximetry during in-vivo calibration experiment with the novel implantable oximeter and comparison with standards. Panel A shows oximetry (StO₂) in porcine models during inspired oxygen modulation in-vivo calibration experiment using the implantable device equipped with an oximeter sensor and commercial tissue oximeter (StO₂—Cutaneous Tissue Ox) and standard blood gas (Blood gas). Panel B is a a correlation curve showing good linearity of the implantable oximeter compared with blood gas (70% Venous:30% Arterial); Correlation coefficient=0.9 (R²), slope of linear regression=0.4. Panel C is a temporal spectrogram that shows the frequency component that corresponds to the heart rate (˜100 bpm) and respiration rate (˜20 Bpm) during the experiment. Panel D shows raw data extracted from the implantable oximeter that show the red and near-infrared (NIR) light channels recorded from the photodiode. The oscillations show the presence of respiration and cardiac pulsation.

In some embodiments, the device further comprises a cartridge module for deployment of the needle in the device.

In some embodiments, the cartridge module is actuated linearly to pierce nearby tissue with the needle or uses a rotational actuation to operate a blade for the deployment of the needle in the device.

In some embodiments, the cartridge module is configured such that the drug delivery mechanism produces hydraulic pressure behind a needle plunger which forces the needle through a protective septum and into the tissue, while simultaneously delivering the drug.

In some embodiments, the cartridge module is equipped with a permanent ring magnet and at least two solenoid coils, wherein a needle penetration force is generated by polarization of the at least two solenoid coils such that the at least two solenoid coils induce repulsive and attractive magnetic forces on the needle magnet, respectively.

In some embodiments, the needle is actuatable repeatedly for multiple piercing events or just once and retracted.

In some embodiments, the cartridge module includes one or more compressed springs for supplying penetration force upon triggering, wherein the one or more compressed springs are held in place with a triggerable stop pin, upon actuation, the one or more compressed springs spring release elastic energy to the needle and forces it into tissue.

In some embodiments, the cartridge module is a spring-loaded needle cartridge with an electromechanical triggering mechanism.

In some embodiments, the cartridge module is strategically located away from the body of the device in surrounding tissue to release drug remotely or integrated directly into the body of the device to allow for a more compact form factor, without requiring internal modification of the cartridge module.

In some embodiments, the at least one electrode comprises copper interdigitated electrodes coated with a bilayer of nickel/gold or other metals such as platinum or nickel.

In some embodiments, the flexible membrane is formed of a flexible material. The flexible material comprises polystyrene-b-polyisoprene-b-polystyrene (SIS), or another mechanically equivalent substitute. In some embodiments, the flexible membrane is a single-layered membrane, or a multilayered membrane. In some embodiments, the flexible membrane is a “smart” membrane equipped with a sensing mechanism that enables monitoring of drug delivery rates via, for example, strain sensing/deformation.

In some embodiments, the delivering member also comprises a plurality of microfluidic channels (or drug outlets) in fluidic communications with the drug exit ports of the at least one drug reservoir (FIGS. 6 and 8).

In some embodiments, the delivering member may also comprise one or more valves in fluidic communications with the drug exit ports of the least one drug reservoir for preventing leakage or accidental release of the drugs. The one or more valves are mechanical or passive valves, or pressure driven float/ball valves. In some embodiments, the one or more valves may comprise breakable seals, and/or elastic septum. In some embodiments, the one or more valves are thermally or electrically activable.

In some embodiments, the delivering member may comprise sheathed hollow needles to for piercing the fibrotic capsule during delivery to ensure fast dosage of rescue drug. The needles comprises microneedles or hypodermic.

In some embodiments, the delivering member comprises one or more sensors to monitor the fill level of drug and electrolyte reservoirs.

In some embodiments, the sensor member comprises at least one optical sensor. The sensor member in some embodiments comprises at least one photoplethysmography (PPG) sensor.

In some embodiments, the sensor member comprises a wireless oximeter for measuring regional tissue oxygen saturation (rStO₂).

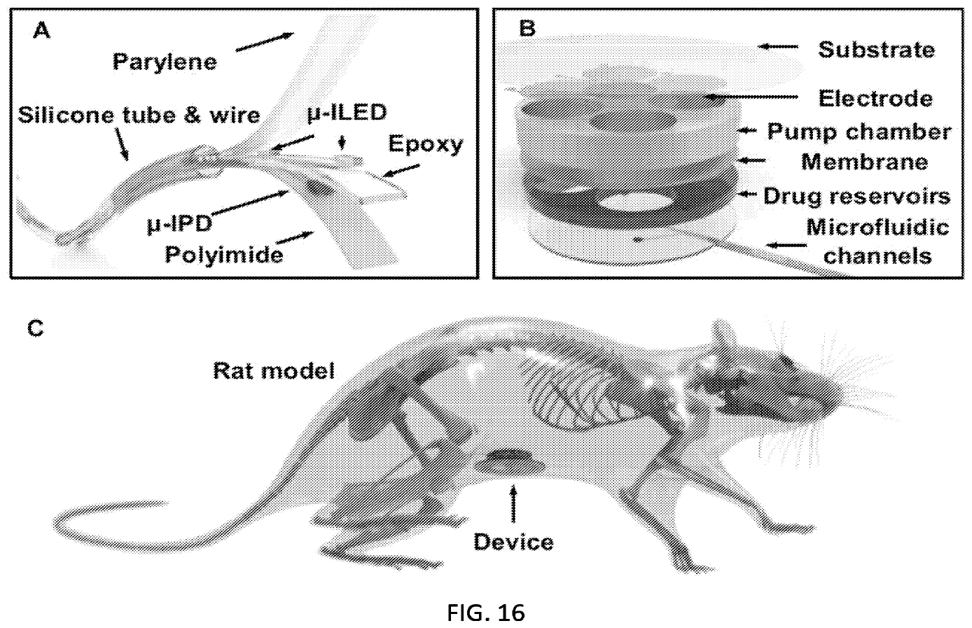

In some embodiments, the oximeter is assembled in the device. In other embodiments, the oximeter is adapted as a peripheral probe. In some embodiments, the oximeter is a low modulus, flexible catheter-type oximetry sensing probe, as shown in panel A of FIG. 16. The low modulus, flexible catheter with an optoelectronic sensor that contains a pair of μ-ILEDs and one μ-IPD allow measurement of deoxygenated and oxygenated hemoglobin, with quantitative accuracy that compares favorably to gold standards. The interconnection and substrate exploits ultrathin, photolithographically defined traces of gold/copper and a flexible polyimide substrate. The measured rStO₂ from such system serves as a metric of respiratory status, for triggering the release of naloxone during pathological suppression of respiration (e.g., overdose).

In some embodiments, the wireless oximeter is untethered from the wireless drug delivery device.

In some embodiments, the sensor member further comprises one or more accelerometers for motion measurements, and/or one or more temperature sensors for temperature measurements and/or ECG electrodes for electrocardiogram measurements.

In some embodiments, the sensor member is configured for multimodal sensing of parameters including SpO₂/StO₂, along with combinations of heart rate, heart rate variability, cardiac sounds, ECG, EMG, activity, body orientation, temperature, blood flow, blood pressure, respiratory rate, respiratory rate variability, respiratory effort/depth, blood chemistry, and subsets of the parameters.

In some embodiments, the sensor member comprises at least two temperature sensors separated by a distance to monitoring local thermal gradients for preventing tissue damage during battery charging.

In some embodiments, the device is configured to have separate and wirelessly connected components including an implant strategically located to measure SpO₂/StO₂ at an optimal body location, and a drug delivery device for drug delivery located at some other location optimized for that purpose. In some embodiments, the components are connected through physical means including wires, tubing, or mechanical structures. In some embodiments, the drug delivery device is configured to deliver one or more drugs at one or more locations simultaneously or sequentially. In some embodiments, the delivered amount of each of the one or more drugs at a respective one of the one or more locations is determined based on the detected physiological status of the living subject.

In some embodiments, the components further comprises a battery that may be located separately from the other components of the device.

In some embodiments, the drug delivery device comprises a booster including integrating supercapacitors or other means to increase the peak power delivery capabilities for accelerating the rates of drug delivery.

In some embodiments, the drug delivery device comprises self-powered pumping mechanisms, wherein the system operably triggers the release of chemical energy through an exothermic chemical reaction, thereby ensuring proper, fast operation of the system even with a depleted battery.

As shown in FIG. 1, the wireless communication system in some embodiments comprises a near field communication (NFC) chip. In some embodiments, the controller is a microcontroller operably in communications with the NFC chip and the sensor member (oximeter) via I2C communication protocol. The controller is configured to initialize measurement of the sensor member, collect the measured data therefrom, and then store the collected data in the NFC chip.

In some embodiments, the implantable device is battery-free, the power for the device is wireless transferred from an external radiofrequency (RF) power source and locally harvested on the device, as shown in FIG. 1. In some embodiments, the power is locally harvested on the devices using a full-wave rectifier, voltage regulator and a supercapacitor bank.

In some embodiments, an NFC reader connected to an external device and in communication with the NFC chip is adapted to provide RF power and communication to power and gain access the device, so as to deliver commands or extract the stored data from the NFC chip. The external device includes, but is not limited to, a smart phone, a tablet, a smart watch, a computer, and so on. In some embodiments, the NFC reader is further adapted to record the sensor member data and perform the data analytics and the closed loop logic of operation.

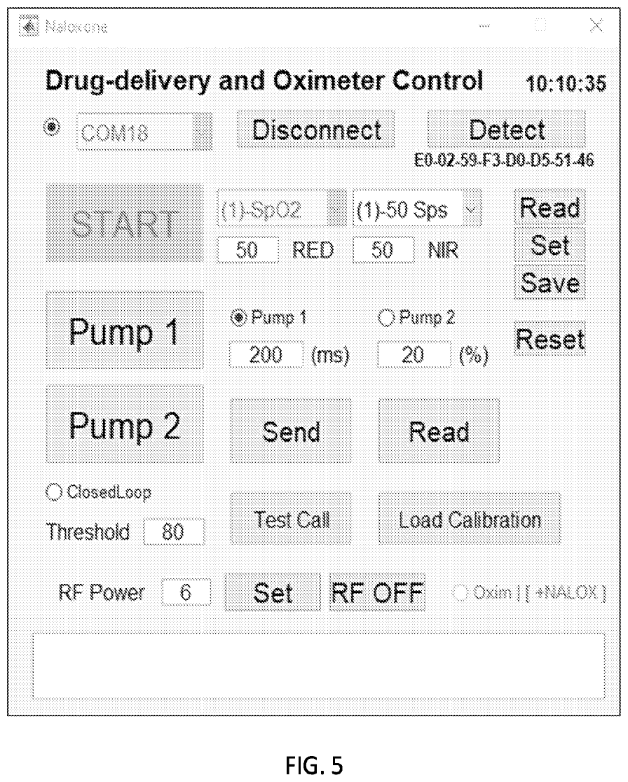

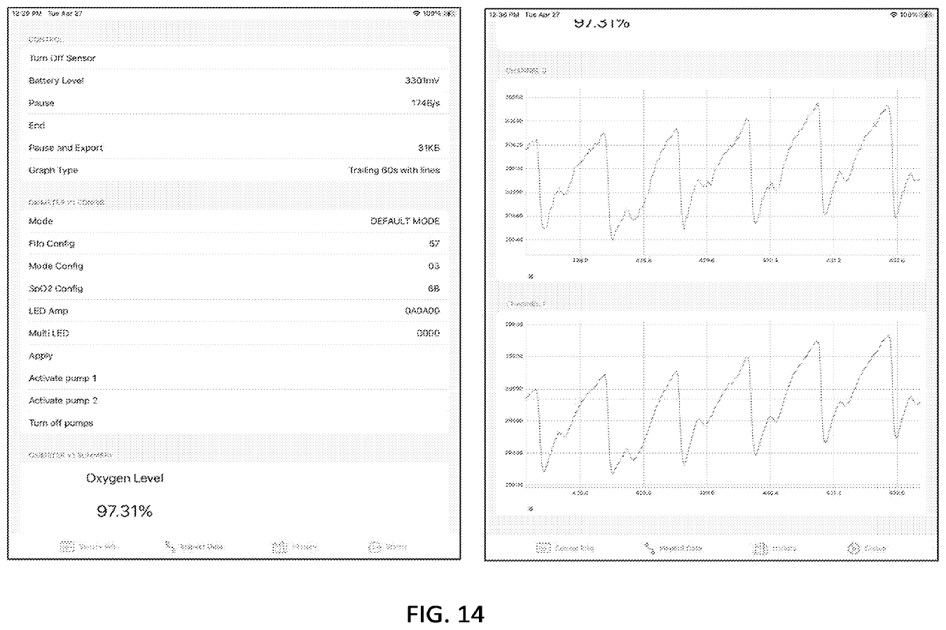

In some embodiments, a customized application with a graphic user interface (GUI) (FIG. 5, or FIG. 14) in the external device is adapted to control the flow of communication with the device via its configuration and operation commands.

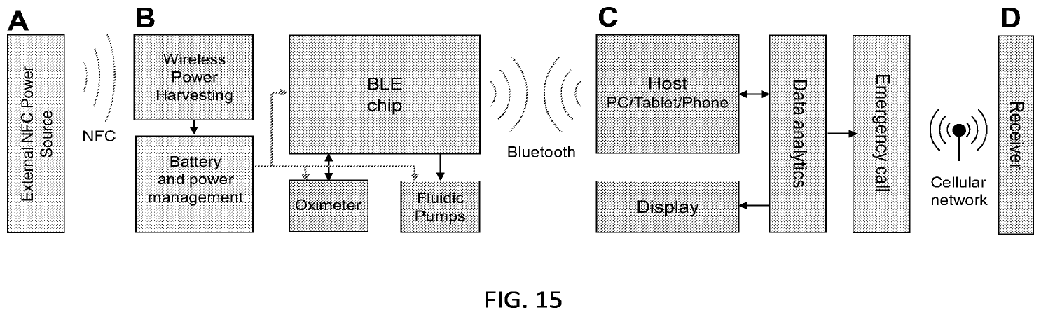

As shown in FIG. 15, the wireless communication system in some embodiments comprises a Bluetooth Low Energy (BLE) module. In some embodiments, the power management system comprises a power module for providing power to the device. The power module may comprise a battery and a battery charging module. The battery in some embodiments is a rechargeable battery. In some embodiments, the battery charging module comprises a transdermal NFC wireless battery charging module. In some embodiments, the battery charging module comprises a smart, intermittent charging algorithm to optimize battery charging while minimizing thermal dissipation at the electronics/tissue interface.

In some embodiments, the power management system is configured such that energy harvesting is from natural body motions, from thermal gradients, from biofuel cells, and the likes.

In some embodiments, the wireless communication system comprises both NFC and BLE communication protocols, optimized for low power operation. In some embodiments, the controller is configured to control the Bluetooth communication with the external device; control the operation of the sensor member via I2C communication protocol; control the activation of either of the two drug delivery pumps; and perform a hybrid power-up mechanism using a combination of firmware, software and passive NFC wireless power transfer, which allows the device to start up even when the battery has drained completely. In some embodiments, a customized application with a graphic user interface (GUI) on the external device is adapted to establish and maintain BLE connection with the devices, control the flow of communication, perform data processing and closed loop logic of operation, log events, and trigger an emergency notice.

In some embodiments, the wireless communication system comprises a cellular or Wi-Fi link for emergency calls or other purposes.

In some embodiments, the device further comprises a biocompatible encapsulation layer that conformally coats entire surrounding of the device. In some embodiments, the encapsulation is coated to prevent the formation of a fibrotic capsule by minimizing the foreign body response.

In some embodiments, the device further comprises flap appendices that are used to secure the device with surgical sutures once implanted.

In some embodiments, the device can be formed in any sizes, preferably in a size less than about 3.5 cm (length)×5 cm (width)×2 cm (height).

In some embodiments, the implantable device is configured to receive inputs from the external device on patient condition or properties of the surroundings or other information streams, and to send data intermittently or continuously to the external device.

In operation, the implantable device receives commands including configuration commands and operation commands. The configuration commands instruct the controller to deploy configuration parameters to the sensor member, or timing configuration for the delivering member. The operation commands trigger activation/deactivation of the sensor member or the delivering member at a time. The configuration commands and the operation commands are initialized from an external device in two-way wireless communications with the controller.

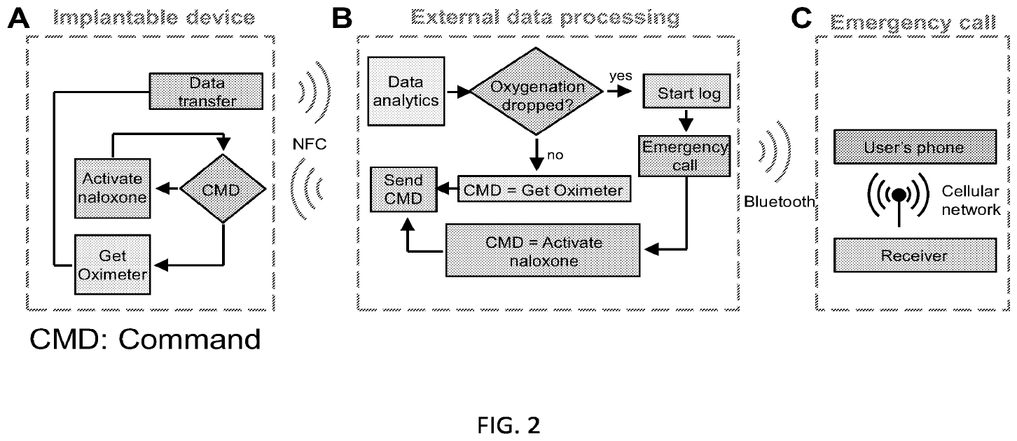

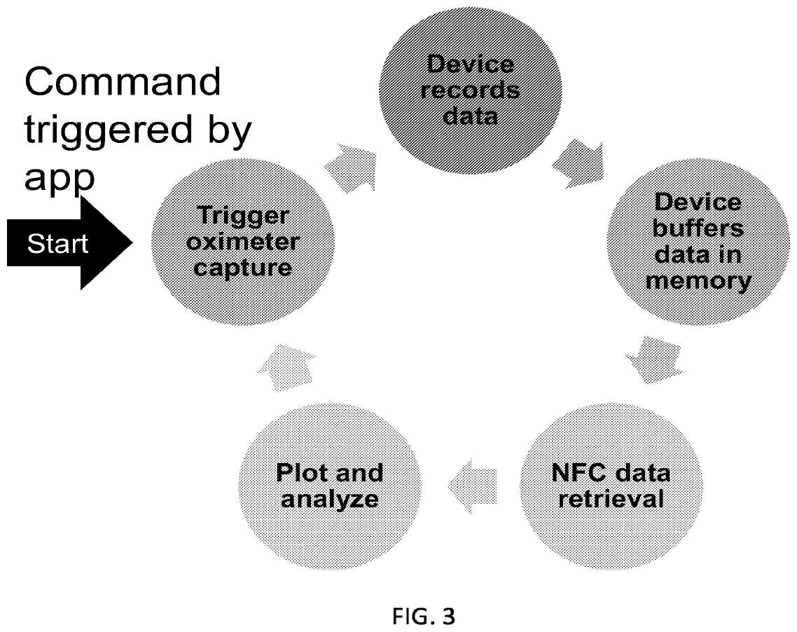

Referring to FIG. 2, a closed-loop logic block diagram of an implantable device is shown according to some embodiments of the invention. As shown in FIG. 3, which shows one embodiment of software operation workflow for the oximeter, initially, “start” command can be sent to the implantable device from an App installed in an external device such as a smartphone, to trigger the oximeter to capture/measure data of the physiological parameters. The device then records the data and buffers the data in memory, for example, in the NFC chip. The NFC data retrieval is transmitted via NFC to the external device for data analysis. When the oxygenation (SpO₂) is dropped below a predetermined threshold value, e.g., 88%, the external device start logs, makes an emergency call and sends a command of “activate naloxone” to the implantable device, which activates naloxone accordingly. When the oxygenation is not dropped below the predetermined threshold value, the external device sends a command of “get oximeter” to the implantable device, which triggers the oximeter to measure the data. The measured data are transferred to the external device for external data processing. As shown in FIGS. 1, 2 and 15, the data transmission between the implantable device and the external device is through the NFC and/or Bluetooth protocols, while the emergency call is the Bluetooth protocol and/or Cellular network.

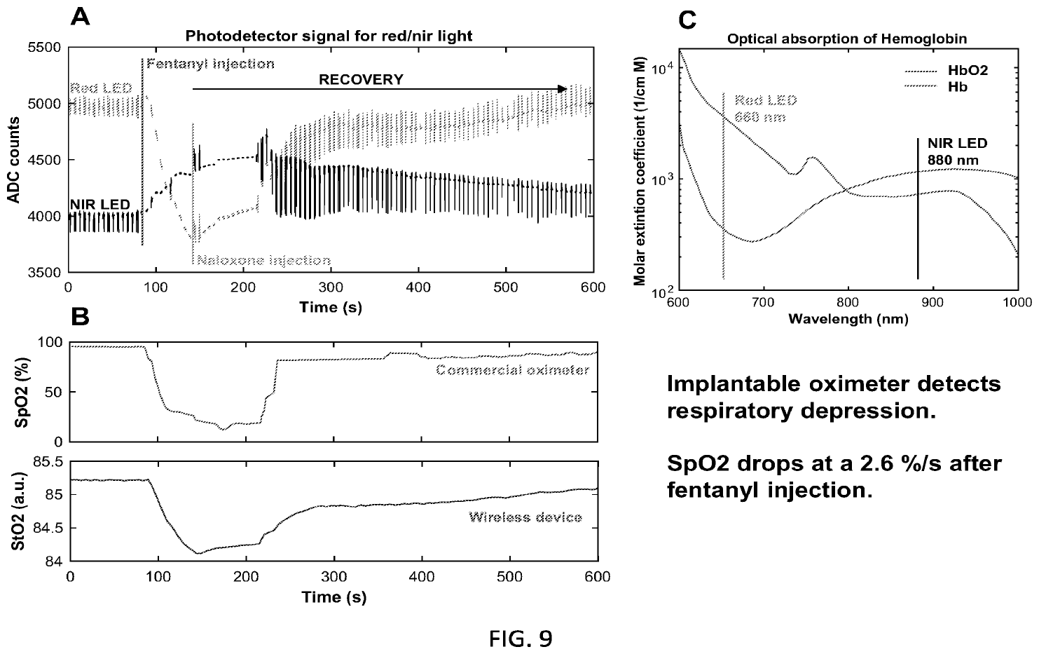

FIG. 9 shows respiratory depression in vivo captured by the optical oximeter of an implantable device according to one embodiment of the invention. A: Raw data recorded by the sensor during respiratory depression. B: Comparison between the implanted device and a commercial oximeter. C: Optical absorption of Hemoglobin at two states; oxygenated hemoglobin (HbO₂) and deoxygenated hemoglobin (Hb).

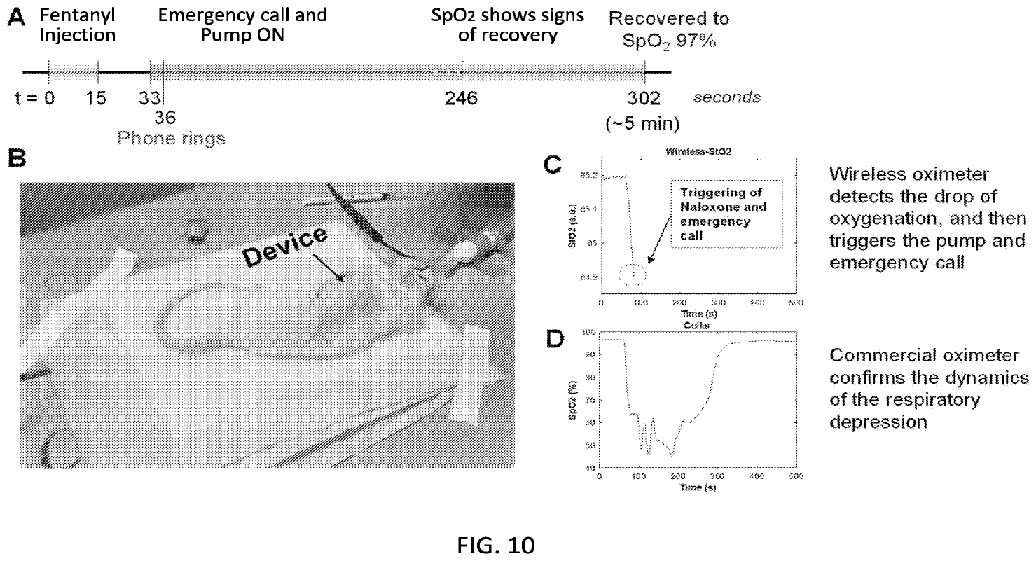

FIG. 10 shows a closed-loop control of respiratory depression using an implantable device in a rat according to one embodiment of the invention. A: Wireless oximeter detects the drop of oxygenation due to fentanyl injection, and then triggers the pump and emergency call. B: Photograph of a rat with a subdermal device. C: Tissue oxygenation recorded by the device showing the event at which it triggers the release of naloxone and the emergency call. D: Commercial oximeter confirms the dynamics of the respiratory depression.

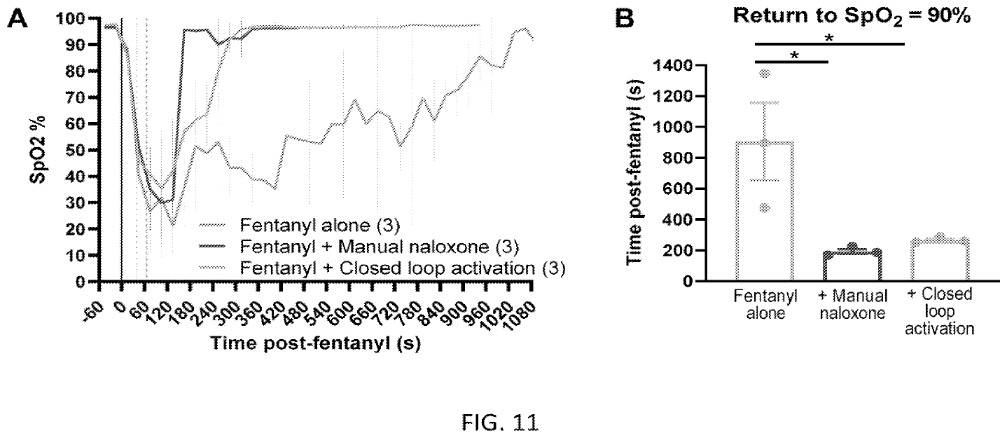

FIG. 11 shows oxygenation traces demonstrating respiratory depression and recovery acquired by an implantable device according to one embodiment of the invention. A: plot comparing the SpO₂ recovery dynamics in-vivo after Fentanyl injection in the following cases: 1) Without naloxone injection, 2) With manual subcutaneous naloxone injection, and 3) With automated naloxone delivery by the implantable device. B: plot showing the elapsed time for the animal model to recover 90% SpO₂. Closed loop administration of naloxone produces comparative recovery as subcutaneous manual injection of naloxone.

In another aspect of the invention, the method for monitoring a physiological status of a living subject and administering drugs therefor includes continuously measuring physiological data of a living subject; processing the physiological data to extract physiological parameters including a heart rate and a tissue oxygenation; determining whether the tissue oxygenation monotonically drop over a period of time based on the physiological parameters; and in the event of an overdose when the tissue oxygenation is lower than a baseline level over the period of time, administering a dose of a drug to the living subject.

In some embodiments, each of the measuring step and the administering step is performed by a device implanted in the living subject, and each of the processing step and the determining step is performed by an external device that is in two-way wireless communication with the device.

In some embodiments, the method further comprises, prior to the processing step, transmitting, by the device, the physiological data to the external device.

In some embodiments, the method further comprises receiving inputs from the external device on patient condition or properties of the surroundings or other information streams; and sending the measured physiological data intermittently or continuously to the external device.

In some embodiments, the method further comprises generating a report of events, by the external device, that include time of event; doses self-administered by the device; the levels of oxygenation recorded before, during and after the overdosing event; and/or geolocation data to be used as a localization resource.

In some embodiments, the method further comprises triggering an emergency notice with geolocation data to first responders in the event of the overdose.

In some embodiments, the method further comprises providing power to the device via a battery.

In some embodiments, the method further comprises wirelessly charging the battery.

In some embodiments, the method further comprises monitoring and controlling safe charging of the battery based on thermal thresholding detected from temperature sensors.

In some embodiments, the method further comprises optimizing operations of the device to prolong battery lifetime and capacity.

In some embodiments, the method further comprises providing power to the device via an NFC chip for power harvesting wirelessly.

In some embodiments, the dose of the at least one drug is determined based on the physiological status, weight, BMI, body temperature, and/or sex of the living subject.

In yet another aspect, the invention also relates to a method for operating continuously and autonomously using hardware/firmware embedded in the implantable device comprising: recording data into memory; making decisions on drug release; generating a report of events; and triggering information transfer and/or emergency calls.

In certain embodiments, the implantable device for monitoring a physiological status of a living subject and administering drugs therefor also includes, but is not limited to, the following additional features, options and aspects.
Valves that can be mechanical or passive to prevent leakage like: breakable seals, float valve, obstruction of passage, thermally activated, electrically activated.
Liquid outlets fitted with adapters to allow for easy connection of tubing for drug delivery.
Redundant microfluidic/drug outlets.
Coatings for preventing the foreign body response and minimize formation of fibrotic capsule.
Microneedle or larger gauge needle for piercing fibrotic capsule during delivery to ensure fast dosage of rescue drug.
Functional membrane equipped with a sensing mechanism to record drug release status.
Sensor to monitor the volume remaining in drug and electrolyte reservoirs.
Smart charging comprising at least two temperature sensors separated at least 1 cm to measure thermal gradient and prevent tissue damage during battery charging.
Intermittent charging method to optimize battery charging while minimizing thermal dissipation in the electronics/tissue interface.
Options for coordinated operation of the implantable device with an external wearable or other (e.g., Alexa, laptop, desktop, etc.) device, in two-way communication, where the implant can receive inputs from the external device on patient condition or properties of the surroundings or other information streams, and where the implant can send data intermittently or continuously.
Aspects of edge computing, in which efficient algorithms operate continuously and autonomously using hardware/firmware embedded in the implantable device—recording data into memory; making decisions on drug release; triggering information transfer and/or emergency calls; etc.
Use of multimodal sensing, e.g., SpO₂/StO₂, along with some combination of heart rate, heart rate variability, cardiac sounds, ECG, EMG, activity, body orientation, temperature, blood flow, blood pressure, respiratory rate, respiratory rate variability, respiratory effort/depth, blood chemistry, etc., and subsets of these parameters.
Configurations in which the implantable device includes separate, wirelessly connected components. For instance, an implant strategically located to measure SpO₂/StO₂ at an optimal body location, and another device for drug delivery located at some other location optimized for that purpose. Two or more devices with this type of strategy.
Configurations like the above, but where the components are connected through physical means, e.g., wires or tubing or mechanical structures. For instance, the battery might be located separately from the other components of the system, as might the SpO₂/StO₂ measuring unit, etc.
Most implantable delivery vehicles delivery drugs at relatively slow speeds. For opioid overdose, speed is key—not only the speed of pumping, but the speed with which the drug enters the blood stream, e.g., considerations in locations of the implant, and locations of the delivery sites into muscle or fat or the vascular system itself.
Options in energy harvesting from natural body motions, from thermal gradients, from biofuel cells, etc.
Options in integrating supercapacitors or other means to increase the peak power delivery capabilities—primarily for accelerating the rates of drug delivery.
Self-powered pumping mechanisms, where the system triggers, for example, the release of chemical energy through an exothermic chemical reaction. In this way, we can ensure proper, fast operation of the system even with a depleted battery.
Options where the cellular or WiFi link for emergency calls or other purposes is built into the implant device itself.
Options in combining both NFC and BLE communication protocols—optimized for low power operations in the device. In addition, different drugs need to be titrated differently for each patient, which are adjustable based on physiological status, weight, BMI, body temperatures, sex, etc.—these parameters dictate different dosing regimens.

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Device Fabrication (Battery-Free Version)

The implantable devices are assembled following standard procedures in electronics technology. The electronic substrate is a printed circuit board (PCB), 0.1 mm thick, fabricated in-house using laser ablation (ProtoLaser U4, LPKF Laser & Electronics) or via outsourced third-party companies. The surface mount components are soldered onto the PCB using hot air and low temperature soldering. A thorough testing of the devices after assembly confirm their successful operation. Then, a bath in isopropanol alcohol cleans soldering residues on the device surface. The drug delivery module is fabricated as follow. The polymeric reservoirs are 3D printed via stereolithography using biocompatible resin (BioMed Amber Resin, Formlabs). The low permeability flexible membrane is composed of polystyrene-b-polyisoprene-b-polystyrene (SIS) block copolymer at 22 wt % styrene. Membranes are fabricated via spincoating (INSTRAS benchtop spinner) SIS/toluene solution on silicon wafer substrates with superhydrophobic coatings and allowed to cure at room temperature for at least 1 hour. Membranes are cut to the desired dimensions using a CO₂ laser. The reservoirs and membrane are assembled with Marine sealant (Marine Adhesive Sealant Fast Cure 5200, 3M). Then this multilayer module is adhered to the PCB, using the same sealant, to form the device.

### Example 2

### Battery-Free Nfc Operation

The electronic system comprises a low power 8-bit microcontroller (Attiny84, Atmel Corporation), a commercial biometric optical sensor (MAX30101, Maxim Integrated Products, Inc.), and a random-access memory (M24LR04E-R, STMicroelectronics) that supports near field communication (NFC). The microcontroller is equipped with a specialized firmware that operates the access to the NFC chip and the optical sensor via I2C communication protocol. Power is transferred wirelessly from an external radiofrequency (RF) power source and locally harvested on the devices using a full-wave voltage rectifier, voltage regulator and a supercapacitor bank. With this configuration the devices support all wireless battery-free operation in experimental enclosures relevant for in-vivo small animal models (rat). These devices contain two drug reservoirs with capacity to enclose 150 μL each of a drug. In some embodiments, this volume can be adjusted in the range of 0.1 to 0.5 mL. The linear dimensions of this device formfactor are 20 mm wide, 30 mm long, and 8 mm thick.

### Example 3

### Bluetooth Operation

The electronic system comprises a low power Arm Cortex M3 microprocessor (MCU, CC2640R2F, Texas Instruments) that supports Bluetooth Low Energy (BLE) 5.1, a commercial biometric optical sensor (MAX30101, Maxim Integrated Products, Inc), and a battery management chip (BMC, BQ25101Y, Texas Instruments). In addition, this device configuration includes transdermal NFC wireless battery charging. These devices contain two drug reservoirs with capacity to enclose 1 mL each of a drug. In some embodiments, this volume is in a range of about 0.5-3 mL. The linear dimensions of this device are 20 mm wide, 30 mm long, and 8 mm thick.

### Example 4

### Drug Delivery Pumps

The devices contain two separate drug delivery reservoirs powered by water electrolysis. Copper interdigitated electrodes (150 μm wide, 150 μm separation), coated with a bilayer of corrosion-resistant Nickel/Gold (4 μm/100 nm), and located on the back of the electronics layer produce electrolysis of an aqueous electrolyte enclosed in the electrolyte reservoir. During water electrolysis, activated by the microcontroller upon an activation command, gas is formed which increases the pressure inside of the electrolyte reservoir. As pressure increases, it deforms the flexible membrane pushing the drug solution out of the reservoir and into the surrounding tissue.

### Example 5

### Device Encapsulation

The devices are doubly encapsulated, first, with a layer of 14 μm thick parylene C that conformally coats the entire device. Then, the device is placed in an acrylic mold designed to fit the device and with produce a smooth and rounded form factor. The mold also includes four flap attachments that are used to secure the devices to tissue with surgical sutures once implanted. Polydimethylsiloxane (PDMS), a biocompatible encapsulant, poured in a multi-step process encapsulates the assembled device with a soft barrier between the body and the device itself. The polymer is cured at 75° C. in multiple steps and finally demolded from the acrylic before punching 1.5 mm holes for suturing using a sterile biopsy punch to complete the process. The linear dimensions of this device are 20 mm wide, 30 mm long, and 8 mm thick.

### Example 6

### Device Operation

The device, regardless of the adopted communication technology (NFC or Bluetooth), receives two types of commands, configuration and operation. Configuration commands instruct the microcontroller/microprocessor to deploy configuration parameters to the optical sensor, or timing configuration for the drug delivery pumps. Operation commands trigger the activation/deactivation of the optical sensor or one of the two drug delivery pumps at a time.

### Example 7

### Battery-Free Operation

During operation of the optical sensor, the microcontroller starts the measurement and collects data, then stores it in the NFC chip (NFC version). The NFC reader (LRM2500-A, Feig Electronic) provides radiofrequency (RF) power and communication to power and gain access the device, so as to deliver commands or extract data from it. This NFC reader is connected to a computer, in which a customized graphic user interface (GUI) implemented in MATLAB (The MathWorks Inc.) controls the flow of communication with the implanted devices via its configuration and operation commands. In addition, the GUI on the computer also records the optical sensor data and performs the data analytics and the closed loop logic of operation.

### Example 8

### Bluetooth Operation

The MCU is equipped with a specialized firmware to perform the following tasks. 1) It controls the Bluetooth communication with an external handheld device. 2) It controls the operation of the optical sensor via I2C communication protocol. 3) It controls the activation of either of the two drug delivery pumps. 4) It performs a hybrid power-up mechanism using a combination of firmware, software and passive NFC wireless power transfer, which that allows the device to start up even when the battery has drained completely. A customized application with a GUI on an iOS device establishes and maintain BLE connection with the implanted devices. This application controls the flow of communication, performs data processing, closed loop logic of operation, log events and trigger an emergency phone call.

### Example 9

### Data Processing and Interpretation

During opioid overdosing the subject will suffer respiratory depression, physiologically observed as a decrease in oxygenation (rStO₂). The device will record oxygenation levels using the optical sensor on a continuous basis. The raw data is transmitted from the implant to a peripheral device (central device) for signal processing. This processing extracts physiological parameters such as heart rate and tissue oxygenation. The implementation of the closed loop is based on tissue oxygenation. Under normal conditions the tissue oxygenation will remain at baseline levels with sporadic fluctuations based on normal daily activities. However, in the event of an overdose, the tissue oxygenation will monotonically drop. Thus, the control algorithm in the GUI detects this physiological event and performs the following tasks. 1) It triggers the release of a life-saving dose of Naloxone, the FDA approved drug to counteract the effect of an opioid overdose. 2) It prepares a report of events that include time of event, doses self-administered by the device, and the levels of oxygenation recorded at the time of overdosing event. 3) It triggers an emergency call to a designated contact. Although automated call to first responders is not permitted in any cellular network provider; however, the integration in the future would be straightforward once signing the appropriate call service contract.

### Example 10

### Exemplary Technical Specifications

The implantable device would resemble a traditional medical device, such as a pacemaker or an implantable drug delivery device. However, the main characteristics are its blood or tissue oxygenation sensing capabilities, the enclosing of a life-saving dose of a naloxone, and the communication with a peripheral hand-held device (smartphone or tablet). In certain embodiments, specific features that constructs these characteristics are detailed below.
Battery lifetime—minimum of 24 hours; wirelessly rechargeable.
Device lifetime about 1 year, or longer.
Blood oxygenation measurements 6 times (or any times) per minute, via in-sensor analytics with PPG.
Heart rate measurements 6 (or any times) times per minute, via in-sensor analytics with PPG.
Respiratory rate measurements 1 time (or any times) per minute, via in-sensor analytics with PPG.
Device traceability via unique ID on the BLE SoC.
Bluetooth Low Energy (BLE) communication to the phone—intermittent, up to once per minute; device operation does not require communication to the phone.
Bluetooth connection distance less than 10 m.
Physician-definable threshold parameters for oxygenation levels and time durations.
Companion application software (Physician) as an interface to the phone to gain access to device configuration.
Companion application software (Patient) as an interface to the phone/mobile device, with event logging, emergency call capability, and GPS access.
   Application monitors the ensemble of signature physiological factors that indicate an opioid-induced respiratory crisis characterized by a sharp, steep decline in rStO2 and representative heart rate and respiratory rate signatures to determine the event of Naloxone triggering.
   Includes an emergency abort option to prevent naloxone delivery due to a false event.
Single or dual drug reservoirs, each with volumes of about 1 mL.
Infusion rate greater than 500 L/min.
Microfluidic channels for release with check valves.
Biocompatible, non-conducting enclosure.
Device size less than 3.5 cm (length)×5 cm (width)×2 cm (height).
Sterilizable via ethylene oxide or radiation (gamma or e-beam).
Refillable reservoirs with cannula.
Reservoir volume options: Each reservoir could be the size of a full preparation (1 or 0.4 mL) or the preparation could be split between 2 reservoirs (0.5 or 0.2 mL):
   0.4 mg/l mL preparation (generics of NARCAN) produced by Hospira, Mylan, International Medication Systems (Amphastar): Could be split between two 500-uL reservoirs, or each reservoir could be filled with 1 mL (800 μg total).
   2 mg/0.4 mL preparation (EVZIO) produced by Kaleo: Each reservoir can be filled with the whole prep, or the prep can be split between 2 reservoirs.
Oximeter sensor on the device, or as a peripheral probe with length of up to about 3 cm.
Drug release through outlets on the device, or through peripheral tubing with length of up to about 3 cm.
Accessible anchoring points for suturing device to tissue as a shallow subdermal implant.
Motion measurements 6 times per minute, via in-sensor analytics via accelerometer.
Temperature measurements 1 time per minute, via embedded temperature sensor.

### Example 11

### Implantable, Wireless Closed-Loop Device, and its Application in Delivery of Naloxone in Rat

In this exemplary example, the device is a fully implantable, wireless pharmacological therapeutic system that combines miniaturized optoelectronic sensors, electrolytic pumps, structures for fluid storage and delivery, and a central control unit, which provides capabilities for highly sensitive, localized measurements of rStO₂ and drug delivery at sites of interest for use in rat models. Specifically, to achieve the desired closed-loop pharmacological intervention, the entire integrated platform may include interconnected sub-systems including, but are not limited to: (1) optoelectronic components, e.g., microscale inorganic light-emitting diodes (μ-ILEDs) and a microscale inorganic photodetector (μ-IPD), on the tip of thin flexible probe for measuring rStO₂ at a site of interest; (2) a collection of drug reservoirs, microfluidic channels, and independently addressable pumping microsystems (total thickness about 4 mm) for fast, triggered deliver of pharmacological agents through a soft probe; (3) a thin, soft base station for bidirectional Bluetooth communication, or near-field communication (NFC), with capabilities for data extraction and user control to facilitate development; (4) a wireless power harvesting unit and collection of supercapacitors to allow uninterrupted operation, without time limit, for freely moving and behaving rats in standard cage enclosures; and (5) a smartphone or tablet computer with customized graphical user interface (GUI) software for real-time visualization of rStO₂ data, equipped with an automated and/or manual control interface to operate closed-loop and/or open-loop interventions. The miniaturized form factors and biocompatible encapsulation approaches associated with each of the implanted sub-systems permit stable, chronic operation in direct measurements of rStO₂ and delivery of drugs into adjacent tissues, in a manner that minimizes mechanically induced irritation and immune responses.

Optical Sensing and Pharmacological Delivery: Both optical sensing and pharmacological delivery modules (sub-systems 1 and 2) electrically connect to the wireless control and power module (WCP, sub-systems 3 and 4). The filament probe in the sensing module exploits optoelectronic designs typical of reflectance-mode, rStO₂, oximeters. Here, a pair of μ-ILEDs (with dimensions of 270 μm×220 μm×50 μm and 240 μm×240 μm×100 μm) and one μ-IPD (with dimensions of 100 μm×100 μm×5 μm) allow measurement of deoxygenated and oxygenated hemoglobin, as shown in panel A of FIG. 16, with quantitative accuracy that compares favorably to gold standards. The interconnection and substrate will exploit ultrathin, photolithographically defined traces of gold/copper (thickness of 700 nm) and a flexible polyimide substrate. The measured rStO₂ from such system serves as a metric of respiratory status, for triggering the release of naloxone during pathological suppression of respiration (e.g., overdose). Specifically, as outlined subsequently, algorithms that identify levels below a threshold value for a prescribed time is used to trigger wirelessly the fast delivery of naloxone contained in microreservoirs (each designed with a volume of 150 μL) via activation of an electrochemical micropump, as shown in panel B of FIG. 16. This micropump and multiple microreservoir system supports low power operation (less than 1 mW), high delivery efficiency (about 90%), large driving force, and multiple injections, all with negligible heating (less than 1° C.) and with chronically stable operation. In this system, electrolysis of an electrolyte solution generates a controlled mechanical force that deforms a flexible membrane toward the drug reservoir, thereby driving the flow of pharmacological agents through the attached, flexible microfluidic probe. The entire system is implanted subcutaneously in the abdomen or other location of a living subject.

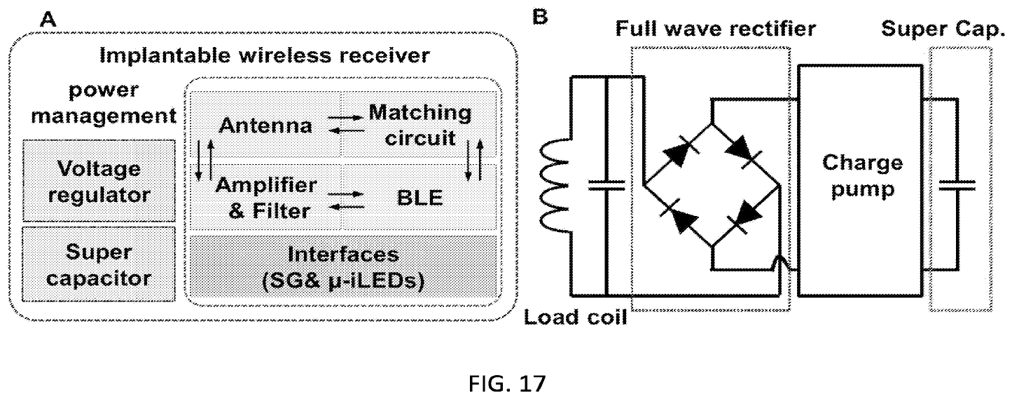

Wireless Control and Power Module (WCP): The WCP manages the overall system power, provides for two-way wireless communication and controls pump operation. In one embodiment, communication relies on the Bluetooth Low Energy (BLE) protocol and uses wireless power management circuitry, as shown in FIG. 17, to harvest energy by magnetic inductive coupling to a transmission coil that encircles the cage. This transmitter incorporates a commercial 13.56 MHz radio frequency identification driver (Feig Electronics Inc.), built-in impedance matching circuitry, and a primary coil antenna. A secondary resonating coil wraps around the bottom of the cage to enhance the power transfer toward the load coil in the WCP. Matching the impedance of the load coils such that they resonate at 13.56 MHz via inductive coupling yields a loosely coupled wireless power transfer system, designed to minimize sensitivities to mismatches between implanted load coil and the fixed primary coils. As a countermeasure for angular mismatches that can arise when the animal rears upward, double-layer supercapacitors integrated into the WCP act as a short-term power buffer (two 3.3V, 80 mF super-capacitors) to allow operation during periods when insufficient power is transferred due to animal movements. These supercapacitors can power the system for more than 3 minutes, continuously, which is more than enough time to power typical disconnection gaps. Furthermore, to protect the device from overheating and Ohmic losses generated by eddy currents, a magnetic shield is placed underneath the IC components and the supercapacitors to redirect the magnetic fields away from the components. The coupled voltage on the load coil is converted using a charge-pump switching regulator, which instantaneously charges and regulates the supercapacitor. A microcontroller embedded with a 2.4 GHz transceiver and an analog to digital converter continuously measures the oxygen level from the response of the μ-IPD to coordinated operation of the pair of μ-ILEDs, and communicates the data to a user interface, for further signal processing. Upon a trigger event, power from the WCP activates one of the 4 electrolytic pumps to deliver a dose of the drug. Up to 4 separate drug injections are possible, if the recovery criteria are not met.

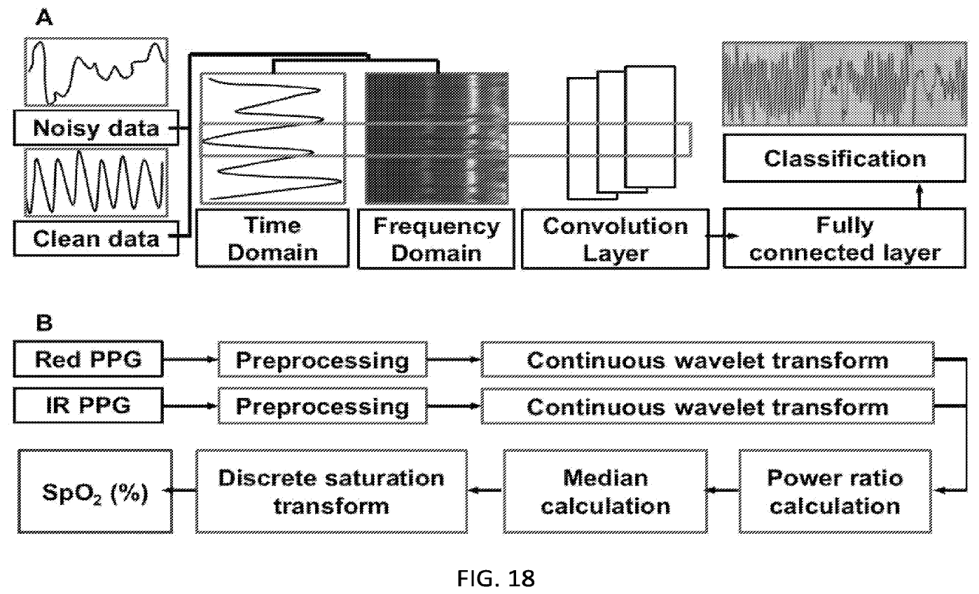

Algorithm: Fully automated, closed-loop operation requires algorithmic accurate, reliable calculation of rStO₂ from data determined with the oximeter. Motion artifacts present the most significant challenges. In one embodiment, a two-stage signal processing approach is implemented, where the first stage determines a metric of signal quality (signal quality index, SQI) of the raw PPG data from the oximeter by utilizing both the time domain and frequency domain representation as a feature for a neural network (NN), as shown in panel A of FIG. 18. This NN is trained with well-established databases (PhysioNet) and with data collected using our own platforms on rat models. The second stage of the signal processing algorithm derives the rStO₂ from the two PPG signals (from the two μ-ILEDs, red and green, the latter of which is selected partly due to the availability of commercial μ-ILEDs at this wavelength) provided that their SQI lies above a set threshold, as shown in panel B of FIG. 18. For this purpose, the continuous wavelet transform (CWT) and the discrete saturation transform (DST) are used in a modified version of the Leeudomwong's algorithm. The first algorithm calculates the rStO₂ with a time-frequency analysis based on the CWT. The DST method provides an additional measure of rStO₂. Agreement between these two values, to within a set tolerance level, provides further confidence in the accuracy. This combined CWT-DST algorithm, together with the SQI approach minimizes false desaturation alarms. Extensive experiments in rat models, with comparisons against our previously-reported oximeter system, shown to yield stable rStO₂ values and trends of variation in vivo, yield data bases to confirm the stability of the proposed algorithms. These studies also establish characteristic timescales for variations in rStO₂ in a range of scenarios, to inform necessary frequency of measurement. These algorithm approaches also yield values of the heart rate (HR) and heart rate variation (HRV). Additionally, the wireless electronic components include built-in temperature sensors. All of these data are captured, for potential use with rStO₂ as additional signatures of health with both redundant and complementary information.

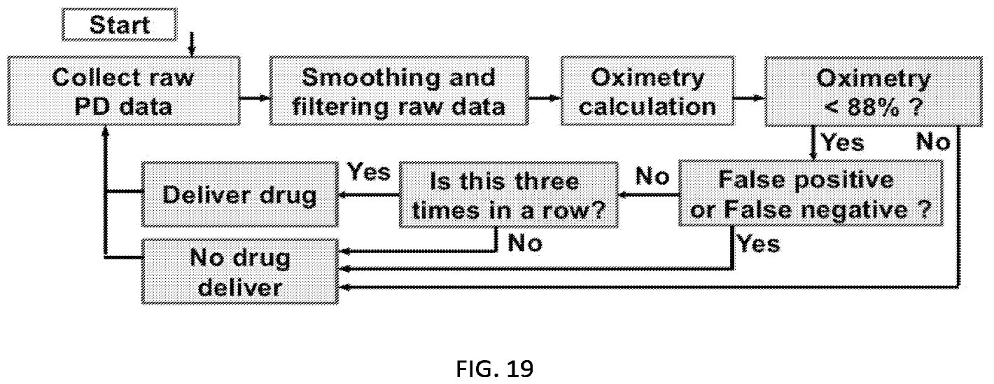

Closed-loop Operation: Closed-loop operation involves development of a software executable that enables real-time processing of the signal, logging of rStO₂ for subsequent analysis, and activation or deactivation of micropump. A user interface allows for an entry point to capture data, adjust set points, and observe performance. Experiments on rat models guide the development of metrics for identifying opioid-induced respiratory depression (decreased rStO₂ level). When the oxygen concentration meets the criteria defined by the input values, e.g., 1 sample per minute, 3 measurements in a row below 88% rStO₂, the system automatically activates one of the micropumps to deliver naloxone, as shown in FIG. 19. A single chamber delivery should be sufficient to reverse the fentanyl effects; however, three reserve chambers enable up to three additional deliveries should the initial delivery fail to return the animal to normoxia. The criteria of three successive readings below 88% rStO₂ are chosen as this is likely to indicate pathological desaturation, and guards against spurious readings initiating a false alarm. It should be appreciated to one skilled in the art that these parameters can and will be adjusted to optimize detection of critical desaturation and minimizing false alarms.

Bluetooth Signalfor Automated Contacting of First Responders: While the naloxone administration initially prevents major harm or death from opioid overdose, naloxone has a short half-life, and the dose may only delay potential harm. Furthermore, the patient is clearly in a situation where medical attention is needed. We therefore include a automated contact to first responders, triggered by the same signal that activates the micropump for naloxone delivery. In one embodiment, an iOS application, developed using XCode, performs all of functions necessary to accomplish these tasks. The app can record oximetry data, define protocols for micropump activation, allow input of calibration parameters for closed-loop operation, and define conditions for the emergency alert call, which is defined as the same conditions that trigger the naloxone injection (e.g., micropump activation). Automated dialing to family and 911 will include delivery of critical information about the caller, including patient information and location; this is needed as the individual in overdose will not be able to talk to the first responders. These auto-dialing and data delivery features, available in currently available apps, will be incorporated into the system control app. Transmission of user-entered demographic information and geolocation via global positioning system (GPS) functionality in the smartphone are included. Although the analytic approach should minimize false desaturation alarms, it is important to provide a mechanism to abort the delivery of drug and initiating the emergency response call in the case of a false signal. In addition, the software will alert the user (vibration and tone) when 2 consecutive measurements detect rStO₂ below 88%. If measurements are not due to overdose, the user can enter a passcode to abort the naloxone injection and emergency call.

Closed-loop naloxone administration and actuation of emergency signal on suppression of rStO₂ following reduced fraction of inspired oxygen (FiO₂). Experiments using hypoxia chambers are the first test of the closed-loop function of the device. With the hypoxia chambers, we can tightly control FiO₂, across a well-defined range, and step down these levels to get a titration curve to demonstrate reliable actuation of the closed-loop system when rStO₂ drops below 88%. The probe of the oximeter is implanted near the femoral artery for in vivo rStO₂ measurements in anesthetized Sprague-Dawley rats (about 300 g). The base station, containing the battery, control hardware, pumping chambers, and drug reservoirs are implanted subcutaneously in the abdomen. The rStO₂ measurements begin after equilibration (−100% O₂ with 2% isoflurane via the nose cone) for 10 min, after which FiO₂ is stepped down gradually to hypoxic conditions (decrease by 5% O₂ every 2 min). rStO₂ is sampled once per minute. On detection of three consecutive readings of rStO₂ below 88%, the closed-loop protocol should activate, and must reliably actuate delivery of naloxone and initiate the warning call to a cell phone coincident with micropump activation. Specifically, the closed-loop operation utilizes a microcontroller embedded with a radio frequency transceiver and an analog-to-digital converter that samples measurements and controls the micropump. A sampling rate of 100 Hz for 3 seconds from the oximeter will yield data for determining the value of rStO₂ that is transmitted to the user-interface device every minute. This low duty cycle operation will conserve battery power to increase the operational lifetime of the system. Additional signal analysis (FIG. 18) via computations through an iOS application will classify the rStO₂ values as either above or below a set threshold to determine when to activate the micropump. The algorithm development section outlines the details of data analysis for accurate oximetry.

Closed-loop naloxone administration and actuation of emergency signal on suppression of rStO₂ following intravenous (i.v) injection of fentanylin rats. The fast-acting and highly potent opioid fentanyl (and analogues) has become the leading cause of overdose deaths. This is due to its relatively high potency and efficacy, but also its relatively simple synthetic pathway, making illicit fentanyl easier and cheaper to make than heroin. Given the opportunity for increased profits, fentanyl has been used as an adulterant in a number of illicit drugs including heroin, cocaine, and more recently, methamphetamine. Over 63,000 Americans died from opioid overdoses in 2016, with more than 19,000 of these directly attributable to fentanyl and it's analogues. Thus, the premise for the use of fentanyl in the present studies is strong, so we focus on fentanyl for our preclinical studies of respiratory depression and overdose. Future studies could test more potent (e.g., carfentanyl) or short acting opioids (e.g., remifentanil, sufentanyl) given the emerging rates of misuse of these drugs.

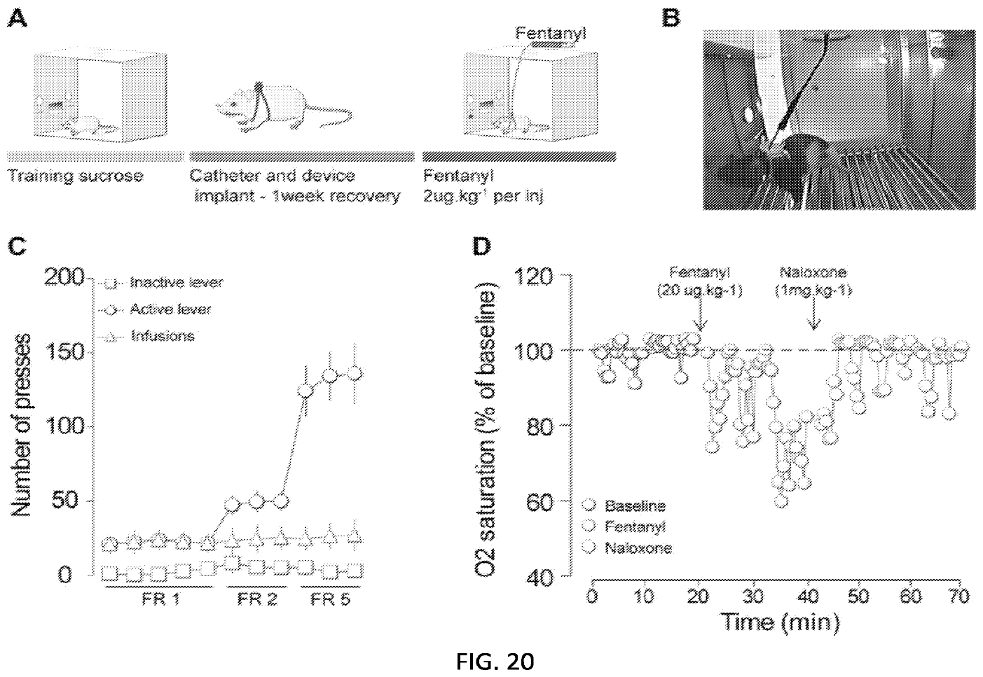

The rat testing involves the injection of fentanyl into awake, freely moving rats via an implanted jugular catheter. We have conducted pilot studies using a commercially-available wearable collar in rats (pane B of FIG. 20) and found that i.v. administration of fentanyl (20 pg/kg) produces respiratory depression and a resulting decrease in rStO2 (panel D of FIG. 20). Unfortunately, the data from these wearable collars are highly impacted by animal movement, producing noisy results, and motivating the device design and advanced analytics. Rats (male and female, 300 g) have the closed-loop system implanted adjacent the femoral artery one week prior to the fentanyl challenge. Rats are freely moving in the exemplary studies. After measurement of baseline parameters, 20 pg/kg fentanyl is injected via the implanted i.v. jugular catheter to induce respiratory depression and reduced rStO₂, and continue monitoring rStO₂ once per minute. On detection of 3 consecutive measurements of rStO₂<88%, the micropump is activated to initiate naloxone administration by the closed-loop protocol, and the device sends a Bluetooth signal to a handheld device for the emergency call. Successful execution will be confirmed by effective reversal of the decreased rStO₂ to normoxia, receipt of the Bluetooth signal on the handheld device, and confirmation post-hoc of delivery of the naloxone solution from the reservoir by visual inspection.

Closed-loop naloxone administration and actuation of emergency signal on suppression of rStO₂ following fentanyl i.v. self-administration in rats. In this phase of the rat study, we test the ability of the device to reverse respiratory depression in rats self-administering fentanyl. To do this, rats (male and female, 300 g at the start of the experiment) first complete a training session in which animals learn to discriminate between active and inactive levers using sucrose (see panel C of FIG. 20 for details). After training, rats are implanted with a jugular catheter. During the same surgery, rats are implanted with the invented wireless closed-loop oximeter placed adjacent the femoral artery (panels A-B of FIG. 20), and the device is secured subcutaneously. A week after surgery, rats are placed back in the self-administration apparatus for 2 hr. Rats seek for reward by pressing on the active lever (panel C of FIG. 20C). Each press results in 2 pg/kg i.v. fentanyl delivery. Cumulative consumption of fentanyl triggers respiratory depression and consequently a decrease in rStO₂. Our device will provide a real-time measurement of this parameter and elicit a subcutaneous naloxone release once the rStO₂ saturation reaches 88% for three consecutive measures (at 1 min intervals). At that time, presses on the lever will not trigger any additional opioid self-administration. Coincident with the closed loop activation of the micropump, the device sends the emergency signal to a handheld device (mimicking a call first responders), as described above. Respiratory parameters are measured for an additional 60 min after naloxone delivery to ensure the full recovery. If the initial injection of naloxone does not effectively reverse respiratory depression within 10 min, additional (up to 4 total) injections of 150 μL naloxone (amounting to 1 mg/kg) are initiated.

Further studies also focus on examination of correlations between rStO₂ and HR, HRV and temperature, to examine the potential use of these additional parameters to increase the robustness and reliability of triggering drug release at appropriate levels of physiological status.

### Example 12

### Actuated Mechanism for Deployment of Needle in Implanted Drug Delivery Device

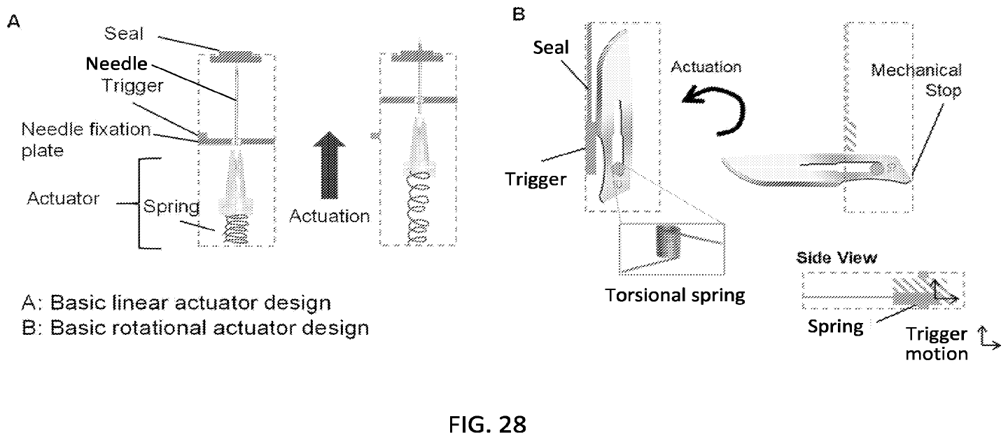

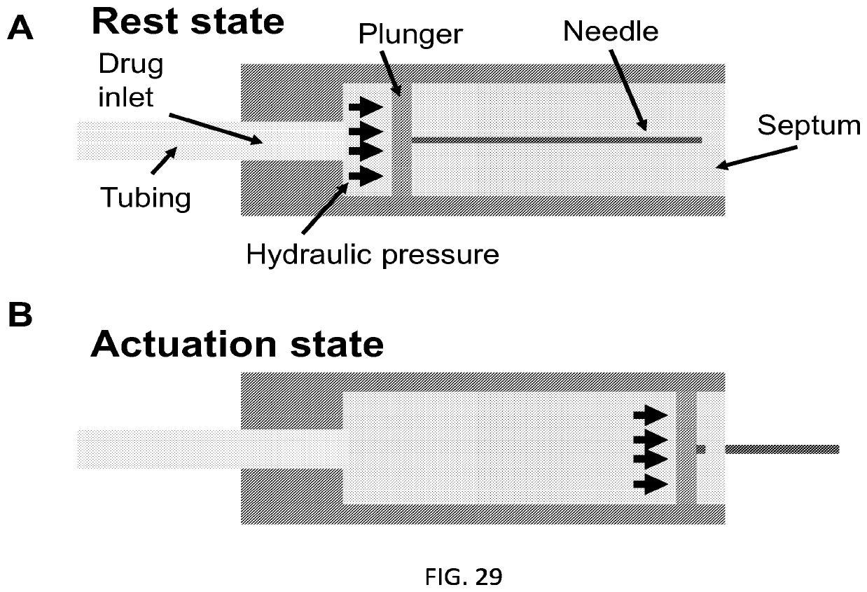

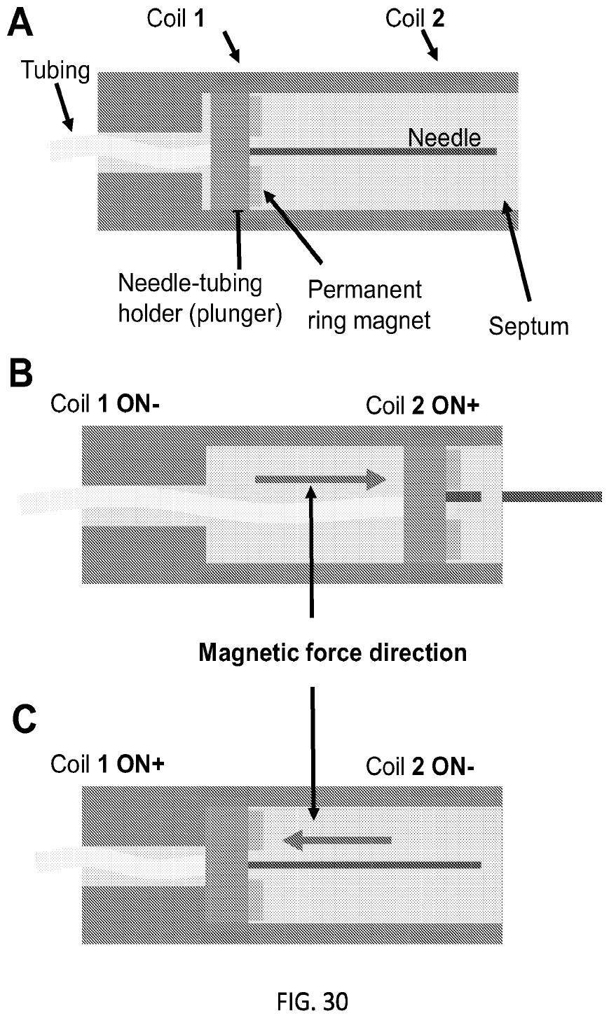

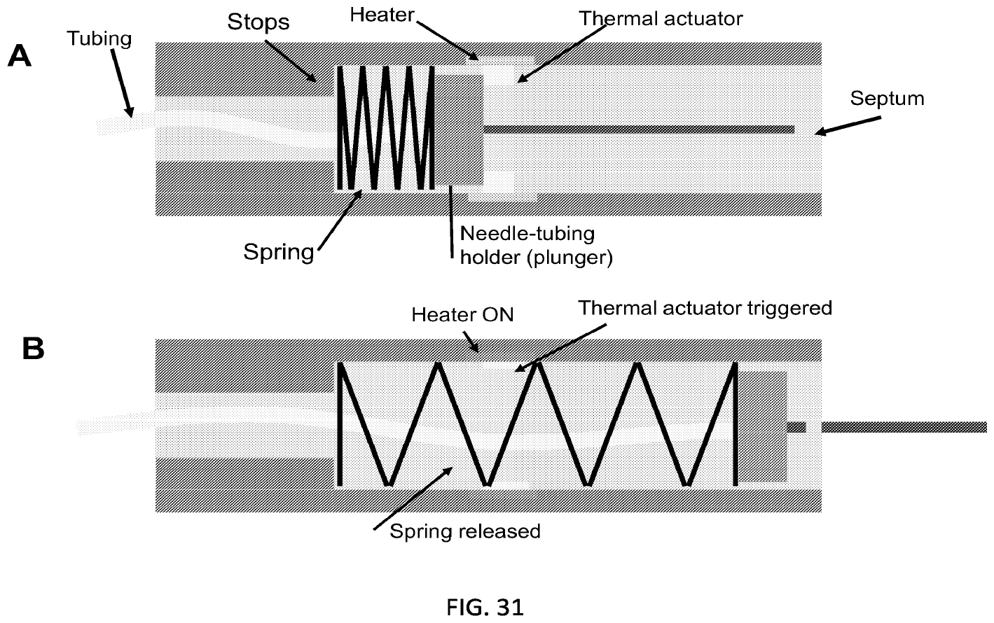

In this exemplary embodiments, the device is equipped with an engineered system to actuate a sharp object with the aim of piercing the avascular fibrous tissue surrounding the implanted device and efficiently delivering the drug to vascularized tissue. This engineered system is referred to as a cartridge module. The cartridge module can be actuated linearly to pierce nearby tissue with a needle, as shown in panel A of FIG. 28, where the cartridge module is equipped with an actuator including a spring, a needle fixation plate for holding the needle and a trigger configured such that when the trigger is triggered, the expansion force of the spring imposed on the needle fixation plate releases the needle fixation plate to move, thereby moving the needle to pierce the seal into a target tissue so as to deliver the drug therein. The cartridge module can also use rotational actuation to operate a blade for the same purpose, as shown in panel B of FIG. 28. Actuation of the cartridge module can be accomplished with several mechanisms. In one example, the drug delivery mechanism produces hydraulic pressure behind a needle plunger which forces the needle through a protective septum and into the tissue, while simultaneously delivering the drug, as shown FIG. 29. In another example, the cartridge module is equipped with a permanent ring magnet and a set of at least two solenoid coils, as shown FIG. 30. In this case, needle penetration force is generated by polarization of both coils such that coil 1 and 2 induce repulsive and attractive magnetic forces on the needle magnet, respectively. To retract the needle back into the housing, a retraction force is generated by polarizing the coils in opposite configuration as the previous case, as shown FIG. 30. In this way, the needle could be actuated repeatedly for multiple piercing events or just once and retracted. In another actuation example, one or more compressed springs is used to supply penetration force upon triggering. In this case, the needle cartridge contains a compressed spring held in place with a triggerable stop pin. Upon actuation, e.g., a heating element, the spring releases elastic energy to the needle and forces it into tissue, as shown FIG. 31.

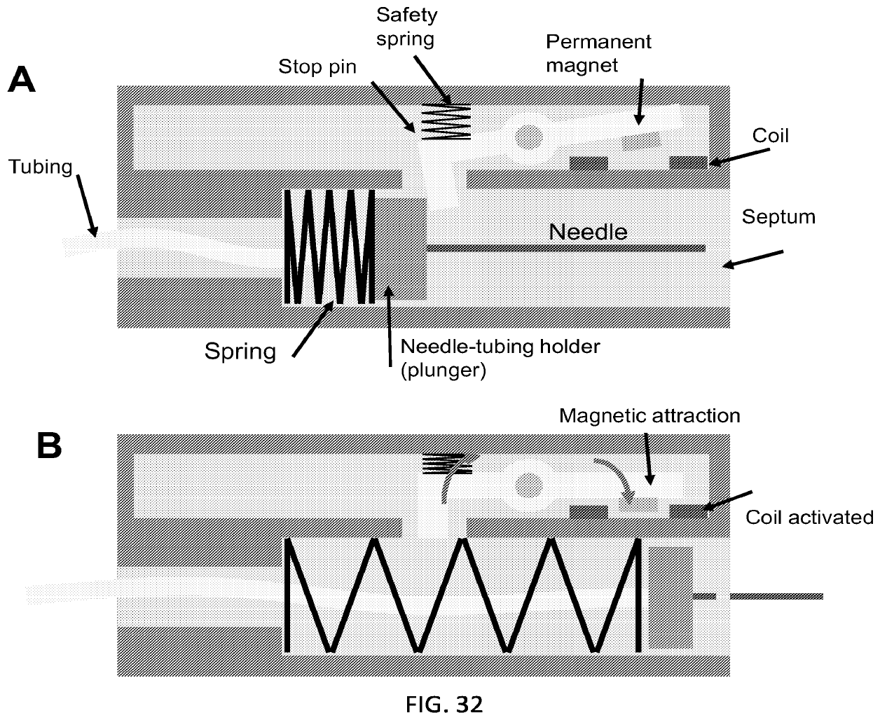

Another example includes a spring-loaded needle cartridge with an electromechanical triggering mechanism, as shown FIG. 32. In this example, the spring is engaged and secured with a stop pin lever. There is a spring on one side of the stop pin to hold it in place and prevent accidental actuation. On the opposite side of the lever, there is a permanent magnet with a concentric coil. When the coil is polarized, an attractive force is produced on the magnet which lifts the stop lever pin restraining the needle and releases the spring.

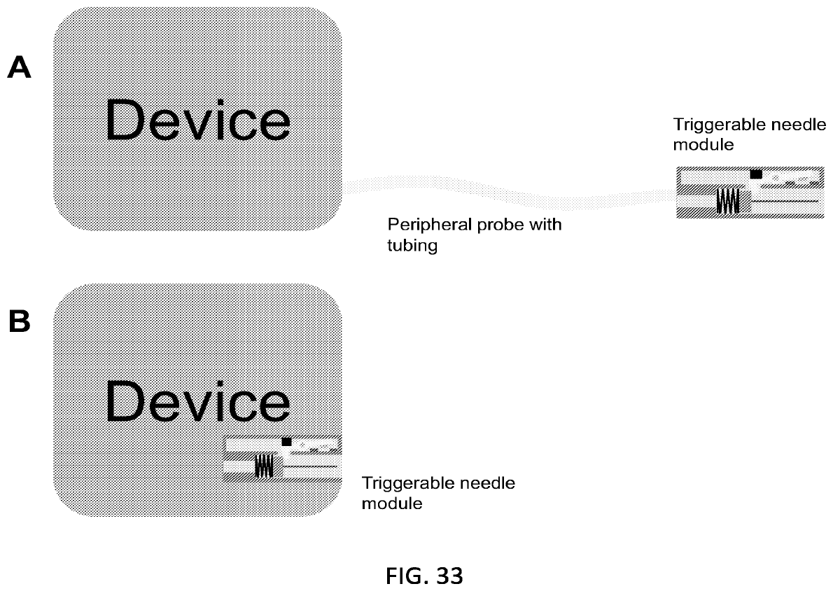

One advantage of the needle cartridge module design is to provide flexibility for incorporating it into multiple device geometries, as shown in FIG. 33. In one case the cartridge can be strategically located away from the body of the device in surrounding tissue to release drug remotely or integrated directly into the body of the device to allow for a more compact form factor, without requiring internal modification of the cartridge design.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. Volkow, N. D., E. B. Jones, E. B. Einstein, and E. M. Wargo, Prevention and Treatment of Opioid Misuse and Addiction: A Review. JAMA Psychiatry, 2019. 76(2): p. 208-216.
[2]. Volkow, N. D. and W. J. Koroshetz, The role of neurologists in tackling the opioid epidemic. Nat Rev Neurol, 2019.
[3]. Comer, S. D. and C. M. Cahill, Fentanyl: Receptor pharmacology, abuse potential, and implications for treatment. Neurosci Biobehav Rev, 2018.
[4]. Strang, J., J. McCambridge, D. Best, T. Beswick, J. Beam, S. Rees, and M. Gossop, Loss of tolerance and overdose mortality after inpatient opiate detoxification: follow up study. BMJ, 2003. 326(7396): p. 959-60.
[5]. Ravndal, E. and E. J. Amundsen, Mortality among drug users after discharge from inpatient treatment: an 8-year prospective study. Drug Alcohol Depend, 2010. 108(1-2): p. 65-9.
[6]. Mickle, A. D., S. M. Won, K. N. Noh, J. Yoon, K. W. Meacham, Y. Xue, L. A. McIlvried, B. A. Copits, V. K. Samineni, K. E. Crawford, D. H. Kim, P. Srivastava, B. H. Kim, S. Min, Y. Shiuan, Y. Yun, M. A. Payne, J. Zhang, H. Jang, Y. Li, H. H. Lai, Y. Huang, S. I. Park, R. W. Gereau, and J. A. Rogers, A wireless closed-loop system for optogenetic peripheral neuromodulation. Nature, 2019. 565(7739): p. 361-365.
[7]. Park, S. I., D. S. Brenner, G. Shin, C. D. Morgan, B. A. Copits, H. U. Chung, M. Y. Pullen, K. N. Noh, S. Davidson, S. J. Oh, J. Yoon, K. I. Jang, V. K. Samineni, M. Norman, J. G. Grajales-Reyes, S. K. Vogt, S. S. Sundaram, K. M. Wilson, J. S. Ha, R. Xu, T. Pan, T. I. Kim, Y. Huang, M. C. Montana, J. P. Golden, M. R. Bruchas, R. W. Gereau, and J. A. Rogers, Soft, stretchable, fully implantable miniaturized optoelectronic systems for wireless optogenetics. Nat Biotechnol, 2015. 33(12): p. 1280-1286.
[8]. Shin, G., A. M. Gomez, R. Al-Hasani, Y. R. Jeong, J. Kim, Z. Xie, A. Banks, S. M. Lee, S. Y. Han, C. J. Yoo, J. L. Lee, S. H. Lee, J. Kurniawan, J. Tureb, Z. Guo, J. Yoon, S. I. Park, S. Y. Bang, Y. Nam, M. C. Walicki, V. K. Samineni, A. D. Mickle, K. Lee, S. Y. Heo, J. G. McCall, T. Pan, L. Wang, X. Feng, T. I. Kim, J. K. Kim, Y. Li, Y. Huang, R. W. Gereau, J. S. Ha, M. R. Bruchas, and J. A. Rogers, Flexible Near-Field Wireless Optoelectronics as Subdermal Implants for Broad Applications in Optogenetics. Neuron, 2017. 93(3): p. 509-521 e3.
[9]. Samineni, V. K., J. Yoon, K. E. Crawford, Y. R. Jeong, K. C. McKenzie, G. Shin, Z. Xie, S. S. Sundaram, Y. Li, M. Y. Yang, J. Kim, D. Wu, Y. Xue, X. Feng, Y. Huang, A. D. Mickle, A. Banks, J. S. Ha, J. P. Golden, J. A. Rogers, and R. W. Gereau, Fully implantable, battery-free wireless optoelectronic devices for spinal optogenetics. Pain, 2017.
[10]. Park, S. I., G. Shin, J. G. McCall, R. Al-Hasani, A. Norris, L. Xia, D. S. Brenner, K. N. Noh, S. Y. Bang, D. L. Bhatti, K. I. Jang, S. K. Kang, A. D. Mickle, G. Dussor, T. J. Price, R. W. Gereau, M. R. Bruchas, and J. A. Rogers, Stretchable multichannel antennas in soft wireless optoelectronic implants for optogenetics. Proc Natl Acad Sci USA, 2016. 113(50): p. E8169-E8177.
[11]. Chung, H. U., B. H. Kim, J. Y. Lee, J. Lee, Z. Xie, E. M. Ibler, K. Lee, A. Banks, J. Y. Jeong, J. Kim, C. Ogle, D. Grande, Y. Yu, H. Jang, P. Assem, D. Ryu, J. W. Kwak, M. Namkoong, J. B. Park, Y. Lee, D. H. Kim, A. Ryu, J. Jeong, K. You, B. Ji, Z. Liu, Q. Huo, X. Feng, Y. Deng, Y. Xu, K. I. Jang, J. Kim, Y. Zhang, R. Ghaffari, C. M. Rand, M. Schau, A. Hamvas, D. E. Weese-Mayer, Y. Huang, S. M. Lee, C. H. Lee, N. R. Shanbhag, A. S. Paller, S. Xu, and J. A. Rogers, Binodal, wireless epidermal electronic systems with in-sensor analytics for neonatal intensive care. Science, 2019. 363(6430).
[12]. Zhang, H., P. Gutruf, K. Meacham, M. C. Montana, X. Zhao, A. M. Chiarelli, A. Vazquez-Guardado, A. Norris, L. Lu, Q. Guo, C. Xu, Y. Wu, H. Zhao, X. Ning, W. Bai, I. Kandela, C. R. Haney, D. Chanda, R. W. Gereau, and J. A. Rogers, Wireless, battery-free optoelectronic systems as subdermal implants for local tissue oximetry. Sci Adv, 2019. 5(3): p. eaaw0873.
[13]. Jeong, J. W., J. G. McCall, G. Shin, Y. Zhang, R. Al-Hasani, M. Kim, S. Li, J. Y. Sim, K. I. Jang, Y. Shi, D. Y. Hong, Y. Liu, G. P. Schmitz, L. Xia, Z. He, P. Gamble, W. Z. Ray, Y. Huang, M. R. Bruchas, and J. A. Rogers, Wireless Optofluidic Systems for Programmable In Vivo Pharmacology and Optogenetics. Cell, 2015. 162(3): p. 662-74.
[14]. Leeudomwong, T., T. Deesudchit, and C. Chinrungruent, Motion-Resistant Pulse Oximetry Processing Based on Time-Frequency Analysis. Engineering Journal, 2017. 21(7): p. 181-196.
[15]. Goldman, J. M., M. T. Petterson, R. J. Kopotic, and S. J. Barker, Masimo signal extraction pulse oximetry. J Clin Monit Comput, 2000. 16(7): p. 475-83.
[16]. Hayashi, K., M. J. Milloy, M. Lysyshyn, K. DeBeck, E. Nosova, E. Wood, and T. Kerr, Substance use patterns associated with recent exposure to fentanyl among people who inject drugs in Vancouver, Canada: A cross-sectional urine toxicology screening study. Drug Alcohol Depend, 2018. 183: p. 1-6.
[17]. Kiyatkin, E. A., Respiratory depression and brain hypoxia induced by opioid drugs: Morphine, oxycodone, heroin, and fentanyl. Neuropharmacology, 2019.
[18]. Hipolito, L., A. Wilson-Poe, Y. Campos-Jurado, E. Zhong, J. Gonzalez-Romero, L. Virag, R. Whittington, S. D. Comer, S. M. Carlton, B. M. Walker, M. R. Bruchas, and J. A. Moron, Inflammatory Pain Promotes Increased Opioid Self-Administration: Role of Dysregulated Ventral Tegmental Area mu Opioid Receptors. J Neurosci, 2015. 35(35): p. 12217-31.
[19]. Swindle, M. M. and M. M. Swindle, Swine in the laboratory: surgery, anesthesia, imaging, and experimental techniques. 2nd ed. 2007, Boca Raton: CRC Press. 471 p.
[20]. Boster, A. L., R. L. Adair, J. L. Gooch, M. E. Nelson, A. Toomer, J. Urquidez, and M. Saulino, Best Practices for Intrathecal Baclofen Therapy: Dosing and Long-Term Management. Neuromodulation, 2016. 19(6): p. 623-31.
[21]. Some, I. T., P. Bogaerts, R. Hanus, M. Hanocq, and J. Dubois, Incorporating batch effects in the estimation of drug stability parameters using an Arrhenius model. Int J Pharm, 1999. 184(2): p. 165-73.
[22]. Chan, D. Y., S. S. Chan, E. K. Chan, A. Y. Ng, A. C. Ying, A. C. Li, C. C. Chiu, N. Cheung, W. K. Mak, D. T. Sun, C. X. Zhu, and W. S. Poon, Blessing or burden?Long-term maintenance, complications and clinical outcome of intrathecal baclofen pumps. Surg Pract, 2018. 22(3): p. 105-110.

1. An implantable device for monitoring a physiological status of a living subject and administering drugs therefor, comprising:
at least one drug reservoir for containing at least one drug solution;
a delivering member coupled to the at least one drug reservoir for operably delivering the at least one drug solution from the at least one drug reservoir to the living subject;
a sensor member for measuring physiological parameters of the living subject so as to monitor a physiological status of the living subject;
a wireless communication system for wireless data transmission;
a power management system for wireless power harvesting; and
a controller coupled to the power management system, the wireless communication system, the sensor member and the delivering member for wireless data transmission and power harvesting; obtaining the physiological status of the living subject, and controlling operations of the delivering member based on the physiological status of the living subject.

2. The device of claim 1, wherein the device operably receives configuration commands and operation commands, wherein the configuration commands instruct the controller to deploy configuration parameters to the sensor member, or timing configuration for the delivering member, and wherein the operation commands trigger activation/deactivation of the sensor member or the delivering member at a time.

3. The device of claim 2, wherein the configuration commands and the operation commands are initialized from an external device in wireless communications with the controller.

4. The device of claim 3, wherein the device is configured to receive inputs from the external device on patient condition or properties of the surroundings or other information streams, and to send data intermittently or continuously to the external device.

5. The device of claim 1, wherein the at least one drug solution comprises naloxone, or other life-saving drugs.

6. The device of claim 1, wherein the at least one drug solution enclosed in the at least one drug reservoir is releasable through drug release outlets on the device, or through peripheral tubing.

7. The device of claim 6, wherein the drug release outlets are fitted with adapters to allow for facile connection to tubing for drug delivery.

8. The device of claim 7, wherein the adapters comprise Luer lock fittings.

9. The device of claim 1, wherein the at least one drug reservoir is refillable.

10. The device of claim 1, wherein the at least one drug reservoir comprises two or more drug reservoirs.

11. The device of claim 10, wherein each of the two or more drug reservoirs contains a same or different drug.

12. The device of claim 10, wherein a dose and a rate of the drug delivery from the two or more drug reservoirs is individually or cooperatively controllable.

13. The device of claim 12, wherein the dose of the drug delivery from the two or more drug reservoirs is determined based on the physiological status, weight, BMI, body temperature, and/or sex of the living subject.

14. The device of claim 10, wherein each of the two or more drug reservoirs has capacity to enclose about 0.1-3 mL of a drug.

15. The device of claim 1, wherein the delivering member comprises a microfluidic drug delivery system.

16. The device of claim 15, wherein the delivering member is powered/operated by water electrolysis.

17. The device of claim 16, wherein the delivering member comprises:
a flexible membrane attached to the at least one drug reservoir;
at least one electrolyte reservoir containing an aqueous electrolyte coupled to the flexible membrane; and
at least one electrode coupled to the at least one electrolyte reservoir for producing the electrolysis of the aqueous electrolyte therein.

18. The device of claim 17, wherein the delivering member is configured such that during the water electrolysis, gas is formed in the at least one electrolyte reservoir, thereby increasing pressure of the at least one electrolyte reservoir, and as the pressure increases, it deforms the flexible membrane pushing the drug solution out of the at least one drug reservoir and into the surrounding tissue of the living subject.

19. The device of claim 17, wherein the at least one electrode comprises copper interdigitated electrodes coated with a bilayer of nickel/gold or other metal including platinum or nickel.

20. The device of claim 17, wherein the flexible membrane is formed of a flexible material.

21. The device of claim 20, wherein the flexible material comprises polystyrene-b-polyisoprene-b-polystyrene (SIS), or similar block copolymer comprised of hard and soft blocks.

22. The device of claim 17, wherein the flexible membrane is a single-layered membrane, or a multilayered membrane.

23. The device of claim 17, wherein the flexible membrane is a smart membrane equipped with a sensing mechanism that enables monitoring of drug delivery rates.

24. The device of claim 23, wherein said monitoring of drug delivery rates is via strain sensing/deformation.

25. The device of claim 17, wherein the at least one electrolyte reservoir comprises:
an effervescent reservoir coupled to the flexible membrane; and
at least one electrolyte chamber containing the aqueous electrolyte, wherein the at least one electrolyte chamber has a gate and is coupled to the effervescent reservoir, wherein the gate is configured to operably open or close the at least one electrolyte chamber such that when the gate is opened, a flow of the aqueous electrolyte into the effervescent reservoir is allowed, and when the gate is closed, the flow of the aqueous electrolyte into the effervescent reservoir is not allowed.

26. The device of claim 25, wherein the flexible membrane is attached to the effervescent reservoir.

27. The device of claim 25, wherein the gate is powered/operated by the electrolysis.

28. The device of claim 25, wherein the at least one electrolyte chamber is filled with citric acid.

29. The device of claim 28, wherein gas is formed in the at least one electrolyte chamber during the electrolysis, thereby increasing the pressure of the at least one electrolyte chamber, and as the pressure increases, it drives the gate to open and pushes the citric acid solution out of the at least one electrolyte chamber and into the effervescent reservoir.

30. The device of claim 28, wherein the effervescent reservoir is filled with sodium bicarbonate (NaHCO₃).

31. The device of claim 30, wherein the at least one drug reservoir comprises a low-friction hollow piston filled with the at least one drug solution.

32. The device of claim 31, wherein the delivering member further comprises a flexible film and a hollow needle attached to the hollow piston.

33. The device of claim 32, wherein the at least one drug solution is releasable through the hollow needle on the device.

34. The device of claim 33, wherein the citric acid reacts with sodium bicarbonate (NaHCO₃) once the gate opens and gas is generated in the effervescent reservoir, thereby increasing the pressure of the effervescent reservoir, and as the pressure increases, it pushes the flexible membrane and the low-friction hollow piston to a block position, while at the same time the fibrous capsule is punched through by the hollow needle, and the drug solution is pushed out from the low-friction piston into the surrounding tissue of the living subject.

35. The device of claim 33, further comprising a cartridge module for deployment of the needle in the device.

36. The device of claim 35, wherein the cartridge module is actuated linearly to pierce nearby tissue with the needle or uses a rotational actuation to operate a blade for the deployment of the needle in the device.

37. The device of claim 35, wherein the cartridge module is configured such that the drug delivery mechanism produces hydraulic pressure behind a needle plunger which forces the needle through a protective septum and into the tissue, while simultaneously delivering the drug.

38. The device of claim 35, wherein the cartridge module is equipped with a permanent ring magnet and at least two solenoid coils, wherein a needle penetration force is generated by polarization of the at least two solenoid coils such that the at least two solenoid coils induce repulsive and attractive magnetic forces on the needle magnet, respectively.

39. The device of claim 38, wherein the needle is actuatable repeatedly for multiple piercing events or just once and retracted.

40. The device of claim 35, wherein the cartridge module includes one or more compressed springs for supplying penetration force upon triggering, wherein the one or more compressed springs are held in place with a triggerable stop pin, upon actuation, the one or more compressed springs spring release elastic energy to the needle and forces it into tissue.

41. The device of claim 35, wherein the cartridge module is a spring-loaded needle cartridge with an electromechanical triggering mechanism.

42. The device of claim 35, wherein the cartridge module is strategically located away from the body of the device in surrounding tissue to release drug remotely or integrated directly into the body of the device to allow for a more compact form factor, without requiring internal modification of the cartridge module.

43. The device of claim 15, wherein the drug delivery system comprises a plurality of microfluidic channel drug outlets in fluidic communications with the least one drug reservoir.

44. The device of claim 15, wherein the drug delivery system comprises one or more valves in fluidic communications with the least one drug reservoir for preventing leakage or accidental release of the drugs.

45. The device of claim 44, wherein the one or more valves are mechanical or passive valves, or pressure driven float/ball valves.

46. The device of claim 44, wherein the one or more valves comprise breakable seals, and/or elastic septum.

47. The device of claim 44, wherein the one or more valves are thermally or electrically activable.

48. The device of claim 15, wherein the drug delivery system comprises sheathed hollow needles for piercing fibrotic capsule during delivery to ensure fast dosage of rescue drug.

49. The device of claim 48, wherein the needles comprises microneedles or hypodermic.

50. The device of claim 15, wherein the drug delivery system comprises one or more sensors to monitor a fill level of the at least one drug reservoir.

51. The device of claim 1, wherein the sensor member comprises at least one optical sensor.

52. The device of claim 51, wherein the sensor member comprises at least one photoplethysmography (PPG) sensor.

53. The device of claim 52, wherein the sensor member comprises a wireless oximeter for measuring regional tissue oxygen saturation (rStO2).

54. The device of claim 53, wherein the oximeter is assembled in the device, or is adapted as a peripheral probe.

55. The device of claim 51, wherein the sensor member further comprises
one or more accelerometers for motion measurements;
one or more temperature sensors for temperature measurements; and/or
ECG electrodes for electrocardiogram measurements.

56. The device of claim 55, wherein the sensor member is configured for multimodal sensing of parameters including SpO₂/StO₂, along with combinations of heart rate, heart rate variability, cardiac sounds, ECG, EMG, activity, body orientation, temperature, blood flow, blood pressure, respiratory rate, respiratory rate variability, respiratory effort/depth, blood chemistry, and subsets of the parameters.

57. The device of claim 51, wherein the sensor member comprises at least two temperature sensors separated by a distance to monitoring local thermal gradients for preventing tissue damage during battery charging.

58. The device of claim 1, wherein the device is configured to have separately and wirelessly connected components including an implant strategically located to measure SpO₂/StO₂ at an optimal body location, and the drug delivery member for drug delivery located at some other location optimized for that purpose.

59. The device of claim 58, wherein the drug delivery member is configured to deliver one or more drugs at one or more locations simultaneously or sequentially.

60. The device of claim 59, wherein the delivered amount of each of the one or more drugs at a respective one of the one or more locations is determined based on the detected physiological status of the living subject.

61. The device of claim 58, wherein the components are connected through physical means including wires, tubing, or mechanical structures.

62. The device of claim 58, wherein the components further comprises a battery that may be located separately from the other components of the device.

63. The device of claim 1, wherein the drug delivery member comprises a booster including integrating supercapacitors or other means to increase peak power delivery capabilities for accelerating the rates of drug delivery.

64. The device of claim 1, wherein the drug delivery member comprises self-powered pumping mechanisms, wherein the power management system operably triggers a release of chemical energy through an exothermic chemical reaction, thereby ensuring proper, fast operation of the device even with a depleted battery.

65. The device of claim 1, wherein the wireless communication system comprises a near field communication (NFC) chip.

66. The device of claim 65, wherein the controller is operably in communications with the NFC chip and the sensor member via I2C communication protocol.

67. The device of claim 65, wherein the power for the device is wirelessly transferred from an external radiofrequency (RF) power source and locally harvested on the device.

68. The device of claim 65, wherein the power is locally harvested on the device using a full-wave rectifier, voltage regulator and a supercapacitor bank.

69. The device of claim 65, wherein the device is battery-free.

70. The device of claim 65, wherein the controller is configured to initialize measurement of the sensor member, collect the measured data therefrom, and store the collected data in the NFC chip.

71. The device of claim 65, wherein an NFC reader that is connected to an external device and in communication with the NFC chip is adapted to provide RF power and communication to power and gain access the device, so as to deliver commands or extract the stored data from the NFC chip.

72. The device of claim 71, wherein a customized application with a graphic user interface (GUI) in an external device is adapted to control the flow of communication with the device via its configuration and operation commands.

73. The device of claim 71, wherein the NFC reader is further adapted to record the sensor member data and perform the data analytics and the closed loop logic of operation.

74. The device of claim 1, wherein the wireless communication system comprises a Bluetooth Low Energy (BLE) module.

75. The device of claim 74, wherein the power management system comprises a power module for providing power to the device.

76. The device of claim 75, wherein the power module comprises a battery and a battery charging module.

77. The device of claim 76, wherein the battery is a rechargeable battery.

78. The device of claim 77, wherein the battery charging module comprises a transdermal NFC wireless battery charging module.

79. The device of claim 76, wherein the battery charging module comprises a smart, intermittent charging algorithm to optimize battery charging while minimizing thermal dissipation at the electronics/tissue interface.

80. The device of claim 1, wherein the power management system is configured such that energy harvesting is from natural body motions, from thermal gradients, from biofuel cells, and the likes.

81. The device of claim 1, wherein the wireless communication system comprises both NFC and BLE communication protocols, optimized for low power operation.

82. The device of claim 81, wherein the controller is configured to
control the Bluetooth communication with an external device;
control the operation of the sensor member via I2C communication protocol;
control the activation of each drug delivery pump; and
perform a hybrid power-up mechanism using a combination of firmware, software and passive NFC wireless power transfer, which allows the device to reset-button-free start up even when the battery has drained completely.

83. The device of claim 82, wherein a customized application with a graphic user interface (GUI) on the external device is adapted to establish and maintain BLE connection with the devices, control the flow of communication, perform data processing, closed loop logic of operation and log events, and trigger an emergency notice.

84. The device of claim 1, wherein the wireless communication system comprises a cellular or Wi-Fi link for emergency calls or other purposes.

85. The device of claim 1, wherein the device has a size less than about 3.5 cm (length)×5 cm (width)×2 cm (height).

86. The device of claim 1, further comprising an encapsulation layer that conformally coats entire surrounding of the device.

87. The device of claim 86, wherein the encapsulation is coated to prevent the formation of a fibrotic capsule by minimizing the foreign body response.

88. The device of claim 1, further comprising reinforced flap appendices that are used to secure the device with surgical sutures once implanted.

89. The device of claim 88, wherein the reinforced flap appendices are used to prevent tear.

90. The device of claim 1, being formed to have smooth and rounded finishing.

91. The device of claim 1, being biocompatible.

92. The device of claim 1, wherein the physiological parameters comprises at least one of a blood oxygenation, a heart rate, a respiratory rate, a temperature, an ECG, and a blood pressure.

93. A method for monitoring a physiological status of a living subject and administering drugs therefor, comprising:
continuously measuring physiological data of a living subject;
processing the physiological data to extract physiological parameters including a heart rate and a tissue oxygenation;
determining whether the tissue oxygenation monotonically drop over a period of time based on the physiological parameters; and
in the event of an overdose when the tissue oxygenation is lower than a baseline level over the period of time, administering a dose of at least one drug to the living subject.

94. The method of claim 93, wherein each of the measuring step and the administering step is performed by a device implanted in the living subject, and wherein each of the processing step and the determining step is performed by an external device that is in two-way wireless communication with the device.

95. The method of claim 94, further comprising, prior to the processing step, transmitting, by the device, the physiological data to the external device.

96. The method of claim 94, further comprising receiving inputs from the external device on patient condition or properties of the surroundings or other information streams; and sending the measured physiological data intermittently or continuously to the external device.

97. The method of claim 94, further comprising:
generating a report of events, by the external device, that include time of event; doses self-administered by the device; the levels of oxygenation recorded before, during and after the overdosing event; and geolocation data to be used as a localization resource.

98. The method of claim 94, further comprising:
triggering an emergency notice with geolocation data to first responders in the event of the overdose.

99. The method of claim 94, further comprising providing power to the device via a battery.

100. The method of claim 99, further comprising wirelessly charging the battery.

101. The method of claim 100, further comprising monitoring and controlling safe charging of the battery based on thermal thresholding detected from temperature sensors.

102. The method of claim 100, further comprising optimizing operations of the device to prolong battery lifetime and capacity.

103. The method of claim 94, further comprising providing power to the device via an NFC chip for power harvesting wirelessly.

104. The method of claim 94, wherein the dose of the at least one drug is determined based on the physiological status, weight, BMI, body temperature, and/or sex of the living subject.

105. A method for operating continuously and autonomously using hardware/firmware embedded in an implantable device according to claim 1, comprising:
recording data into memory;
making decisions on drug release based on the recorded data;
generating a report of events; and
triggering information transfer and/or emergency calls.
